(19) United States
(12) Patent Application Publication
(10) Pub. No.: US 2025/0134462 A1
(43) Pub. Date: May 1, 2025

(54) DETECTION DEVICE AND WEARABLE DEVICE

(71) Applicants: Japan Display Inc., Tokyo (JP); The University of Tokyo, Tokyo (JP)

(72) Inventors: Kento Himoto, Tokyo (JP); Takashi Nakamura, Tokyo (JP); Takao Someya, Tokyo (JP); Tomoyuki Yokota, Tokyo (JP); Dongkai Cheng, Tokyo (JP)

(21) Appl. No.: 19/009,680

(22) Filed: Jan. 3, 2025

Related U.S. Application Data
(63) Continuation of application No. PCT/JP2023/024123, filed on Jun. 29, 2023.

(30) Foreign Application Priority Data
Jul. 8, 2022 (JP) 2022-110345

(51) Int. Cl. A61B 5/00; A61B 5/1455

(52) U.S. Cl. CPC A61B 5/6826; A61B 5/14552; A61B 5/6824; A61B 5/6828; A61B 5/7225; A61B 2562/0233

(57) ABSTRACT

According to an aspect, a detection device includes: a plurality of optical sensors arranged in a detection area; a light source configured to emit light to the optical sensors; an analog front-end (AFE) circuit configured to acquire a detection value of each of the optical sensors; and a signal processing circuit configured to acquire predefined biometric information based on first time-domain data obtained by acquiring the detection values in chronological order. The signal processing circuit is configured to: convert the first time-domain data into a time-domain matrix and perform singular value decomposition on the time-domain matrix, and inversely calculate second time-domain data based on a predetermined singular value among a plurality of singular values obtained as a result of the singular value decomposition; and acquire the biometric information that changes in chronological order as image information using the second time-domain data.

### CROSS-REFERENCE TO RELATED APPLICATION

This application claims the benefit of priority from Japanese Patent Application No. 2022-110345 filed on Jul. 8, 2022 and International Patent Application No. PCT/JP2023/024123 filed on Jun. 29, 2023, the entire contents of which are incorporated herein by reference.

### BACKGROUND

### 1. Technical Field

What is disclosed herein relates to a detection device and a wearable device.

### 2. Description of the Related Art

United States Patent Application Publication No. 2018/0012069 describes an optical sensor in which a plurality of photoelectric conversion elements such as photodiodes are arranged on a semiconductor substrate. With the optical sensor, signals output from the photoelectric conversion elements change with an amount of received light, thereby enabling detection of biometric information.

Japanese Patent Application Laid-open Publication No. 2019-180861 describes a configuration to acquire an oxygen saturation level in blood (hereinafter, called “blood oxygen saturation level” (SpO₂)) using pulse waves acquired using infrared light and pulse waves acquired using red light. The blood oxygen saturation level (SpO₂) refers to a ratio of an amount of oxygen actually bound to hemoglobin to the total amount of oxygen under the assumption that the oxygen is bound to all the hemoglobin in the blood.

For example, when acquiring subcutaneous information such as the pulse waves and blood flow, appropriate biometric information may not be obtained due to superimposition of body motion noise caused by human movement or the like, biosignals not intended to be detected, or noise components at an alternating-current frequency of a commercial power supply (for example, 50 Hz or 60 Hz).

For the foregoing reasons, there is a need for a detection device and a wearable device that are capable of acquiring desired biometric information.

### SUMMARY

According to an aspect, a detection device includes: a plurality of optical sensors arranged in a detection area; a light source configured to emit light to the optical sensors; an analog front-end (AFE) circuit configured to acquire a detection value of each of the optical sensors; and a signal processing circuit configured to acquire predefined biometric information based on first time-domain data obtained by acquiring the detection values in chronological order. The signal processing circuit is configured to: convert the first time-domain data into a time-domain matrix and perform singular value decomposition on the time-domain matrix, and inversely calculate second time-domain data based on a predetermined singular value among a plurality of singular values obtained as a result of the singular value decomposition; and acquire the biometric information that changes in chronological order as image information using the second time-domain data.

According to an aspect, a wearable device includes the detection device, and the wearable device has a ring shape wearable on a human body.

### BRIEF DESCRIPTION OF THE DRAWINGS

FIG. 1 is a plan view illustrating a detection device according to an embodiment;

FIG. 2 is a block diagram illustrating a configuration example of the detection device according to the embodiment;

FIG. 3 is a circuit diagram illustrating the detection device according to the embodiment;

FIG. 4 is a circuit diagram illustrating a plurality of partial detection areas of the detection device according to the embodiment;

FIG. 5 is a schematic partial sectional view of an optical sensor according to the embodiment;

FIG. 6 is a timing waveform diagram illustrating an operation example of the detection device according to the embodiment;

FIG. 7 is a timing waveform diagram illustrating an operation example in a reset period in FIG. 6;

FIG. 8 is a timing waveform diagram illustrating an operation example in a readout period in FIG. 6;

FIG. 9 is a timing waveform diagram illustrating an operation example in a drive period for one gate line included in the readout period in FIG. 6;

FIG. 10 is an explanatory diagram for explaining a first example of a relation between driving of a sensor area and lighting operations of light sources in the detection device according to the embodiment;

FIG. 11 is a second explanatory diagram for explaining a second example of the relation between driving of the sensor area and the lighting operations of the light sources in the detection device according to the embodiment;

FIG. 12 is a timing waveform diagram illustrating an operation example in the second example illustrated in FIG. 11;

FIG. 13 is a schematic view illustrating a device representing a first application example of the detection device according to the embodiment;

FIG. 14 is a schematic view illustrating a device representing a second application example of the detection device according to the embodiment;

FIG. 15 is a flowchart illustrating an exemplary process in a signal processing circuit of the detection device according to the embodiment;

FIG. 16 is an illustrative diagram of time-domain data acquired in a detection area in a predetermined period;

FIG. 17 is a conceptual diagram for explaining an outline of a singular value decomposition process;

FIG. 18 is a waveform diagram illustrating an exemplary pulse wave;

FIG. 19 is an illustrative diagram illustrating exemplary frequency components included in the pulse wave;

FIG. 20 is an illustrative diagram illustrating an exemplary frequency distribution obtained by applying a fast Fourier transform (FFT) process to the time-domain data constituting the waveform;

FIG. 21 is an illustrative diagram of the FFT process;

FIG. 22 illustrates illustrative diagrams of a process using the singular value decomposition according to the embodiment;

FIG. 23 illustrates illustrative diagrams illustrating exemplary frequency components decomposed by the singular value decomposition process according to the embodiment; and

FIG. 24 illustrates illustrative diagrams illustrating exemplary biometric information acquired as image information by the detection device according to the embodiment.

### DETAILED DESCRIPTION

The following describes a mode (embodiment) for carrying out the present disclosure in detail with reference to the drawings. The present disclosure is not limited to the description of the embodiment given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present specification and the drawings, and detailed description thereof may not be repeated where appropriate.

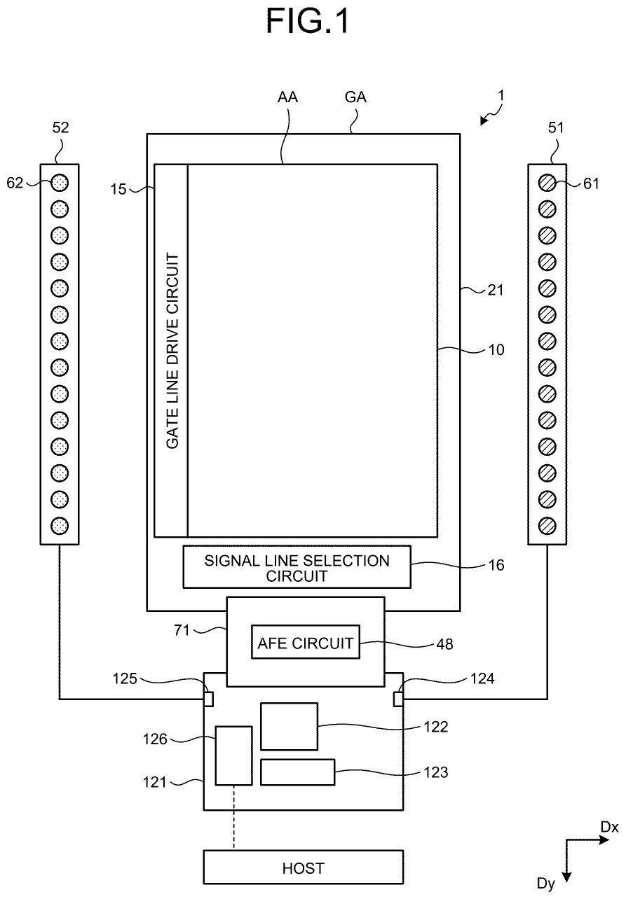

FIG. 1 is a plan view illustrating a detection device according to an embodiment. As illustrated in FIG. 1, a detection device 1 includes a sensor base member 21, a sensor area 10, a gate line drive circuit 15, a signal line selection circuit 16, an analog front-end (AFE) circuit 48, a control circuit 122, a power supply circuit 123, first light sources 61, and second light sources 62. FIG. 1 illustrates an example in which a first light source base member 51 is provided with a plurality of the first light sources 61, and a second light source base member 52 is provided with a plurality of the second light sources 62. However, the arrangement of the first and the second light sources 61 and 62 illustrated in FIG. 1 is merely exemplary and can be modified as appropriate. For example, the first and the second light sources 61 and 62 may be arranged on each of the first and the second light source base members 51 and 52. In this case, a group including the first light sources 61 and a group including the second light sources 62 may be arranged in a second direction Dy, or the first and the second light sources 61 and 62 may be alternately arranged in the second direction Dy. The first and the second light sources 61 and 62 may be provided on one light source base member, or three or more light source base members. A specific example of the arrangement of the first and the second light sources 61 and 62 will be described later.

The detection device 1 is electrically coupled to a host. The host is, for example, a higher-level control device for an apparatus (not illustrated) to which the detection device 1 is applied. The detection device 1 according to the first embodiment transmits acquired biometric information to the host via an output circuit 126.

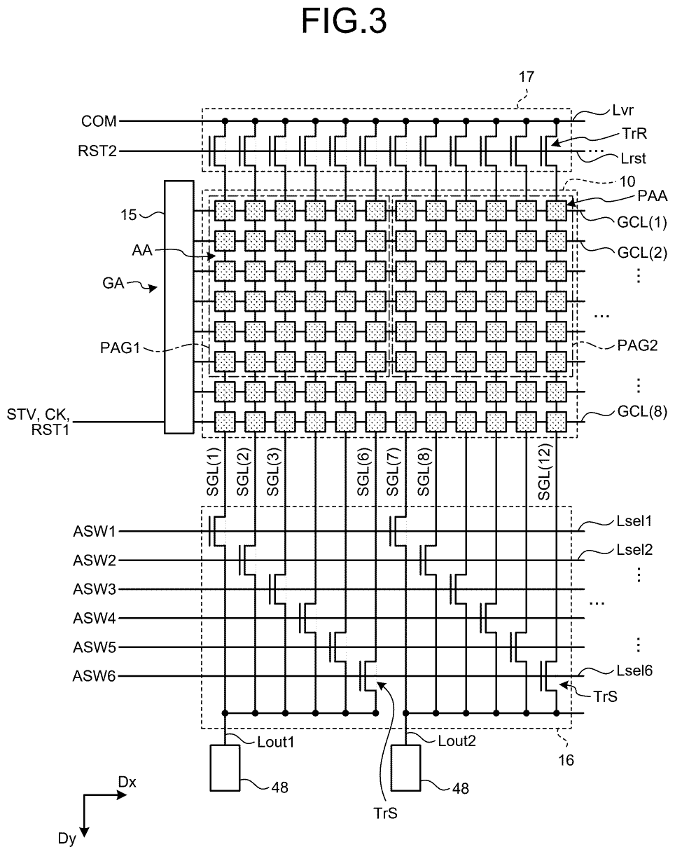

The sensor base member 21 is electrically coupled to a control substrate 121 via a flexible printed circuit board 71. The flexible printed circuit board 71 is provided with the AFE circuit 48. The control substrate 121 is provided with the control circuit 122, the power supply circuit 123, and the output circuit 126. As illustrated in FIG. 3 to be described later, the AFE circuit 48 is circuitry including a plurality of AFE circuits each of which is provided for a plurality signal lines. In the following descriptions, each of the plurality of AFE circuits included in the AFE circuit 48 as entire circuitry is given the same reference sign “48” and is referred to as the “AFE circuit 48” in some cases.

The control circuit 122 is, for example, a control integrated circuit (IC) that outputs logic control signals. The control circuit 122 may be, for example, a programmable logic device (PLD) such as a field-programmable gate array (FPGA).

The control circuit 122 supplies control signals to the sensor area 10, the gate line drive circuit 15, and the signal line selection circuit 16 to control detection operations in the sensor area 10. The control circuit 122 also supplies control signals to the first and the second light sources 61 and 62 to control lighting and non-lighting of the first and the second light sources 61 and 62.

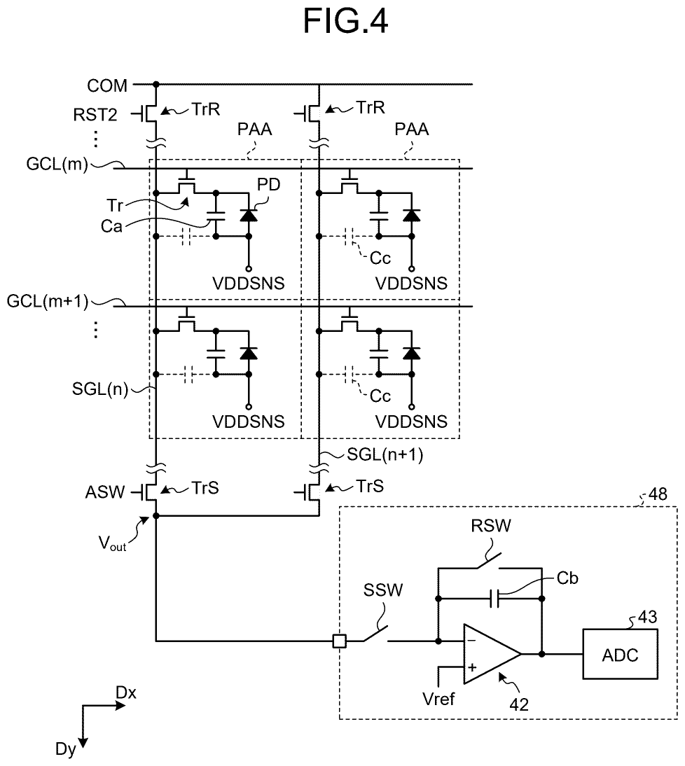

The power supply circuit 123 supplies voltage signals such as a sensor power supply potential VDDSNS (refer to FIG. 4) to the sensor area 10, the gate line drive circuit 15, and the signal line selection circuit 16. The power supply circuit 123 supplies a power supply voltage to the first and the second light sources 61 and 62.

The output circuit 126 is, for example, a Universal Serial Bus (USB) controller IC, and controls communication between the control circuit 122 and the host.

The sensor base member 21 has a detection area AA and a peripheral area GA. The detection area AA is an area where a plurality of optical sensors PD (refer to FIG. 4) included in the sensor area 10 are provided in a matrix having a row-column configuration. The peripheral area GA is an area between the outer perimeter of the detection area AA and the ends of the sensor base member 21, and is an area not provided with the optical sensors PD.

The gate line drive circuit 15 and the signal line selection circuit 16 are provided in the peripheral area GA. Specifically, the gate line drive circuit 15 is provided in an area extending along the second direction Dy in the peripheral area GA. The signal line selection circuit 16 is provided in an area extending along a first direction Dx in the peripheral area GA, and is provided between the sensor area 10 and the AFE circuit 48.

The first direction Dx is one direction in a plane parallel to the sensor base member 21. The second direction Dy is one direction in the plane parallel to the sensor base member 21 and is a direction orthogonal to the first direction Dx. The second direction Dy may non-orthogonally intersect the first direction Dx. A third direction Dz is a direction orthogonal to the first direction Dx and the second direction Dy and is a direction normal to the sensor base member 21.

The first light sources 61 are provided on the first light source base member 51 and arranged along the second direction Dy. The second light sources 62 are provided on the second light source base member 52, and arranged along the second direction Dy. The first light source base member 51 and the second light source base member 52 are electrically coupled to the control circuit 122 and the power supply circuit 123, through terminals 124 and 125, respectively, provided on the control substrate 121.

For example, inorganic light-emitting diodes (LEDs) or organic electroluminescent (EL) diodes (organic light-emitting diodes (OLEDs)) are used as the first and the second light sources 61 and 62. The first and the second light sources 61 and 62 emit first and second light, respectively, having different wavelengths.

The first light emitted from the first light sources 61 is reflected, for example, on a surface of an object to be detected, such as a finger or a wrist of a subject of examination, and is incident on the sensor area 10. As a result, the sensor area 10 can detect a fingerprint by detecting a shape of asperities on the surface of a finger Fg or the like. The second light emitted from the second light sources 62 is, for example, reflected in the finger Fg or the like, or transmitted through the finger Fg or the like, and is incident on the sensor area 10. As a result, the sensor area 10 can detect information on a living body in the finger, the wrist, or the like of the subject of examination. Examples of the information on the living body include, but are not limited to, pulse waves, pulsation, and a vascular image of the subject of examination. That is, the detection device 1 may be configured as a fingerprint detection device that detects a fingerprint or a vein detection device that detects a vascular pattern of, for example, veins.

The first light may have a wavelength of 420 nm to 600 nm, for example, approximately 500 nm, and the second light may have a wavelength of 780 nm to 950 nm, for example, approximately 850 nm. In this case, the first light is blue or green visible light (blue light or green light), and the second light is infrared light. The sensor area 10 can detect a fingerprint based on the first light emitted from the first light sources 61. The second light emitted from the second light sources 62 is reflected in, or transmitted through or absorbed by the object to be detected, and is incident on the sensor area 10. As a result, the sensor area 10 can detect biometric data such as the pulse waves and the vascular image (vascular pattern) as the information on the living body in the finger, the wrist, or the like of the subject of examination.

Alternatively, the first light may have a wavelength of 600 nm to 700 nm, for example, approximately 660 nm, and the second light may have a wavelength of 780 nm to 950 nm, for example, approximately 850 nm. In this case, the sensor area 10 can detect a blood oxygen level in addition to the pulse waves, the pulsation, and the vascular image as the information on the living body based on the first light emitted from the first light sources 61 and the second light emitted from the second light sources 62. In this way, the detection device 1 includes the first and the second light sources 61 and 62, and performs the detection based on the first light and the detection based on the second light, and thereby can detect the various types of information on the living body. The emission colors of the first and the second light sources 61 and 62 described above are examples, and the present disclosure is not limited by the emission colors of the first and the second light sources 61 and 62.

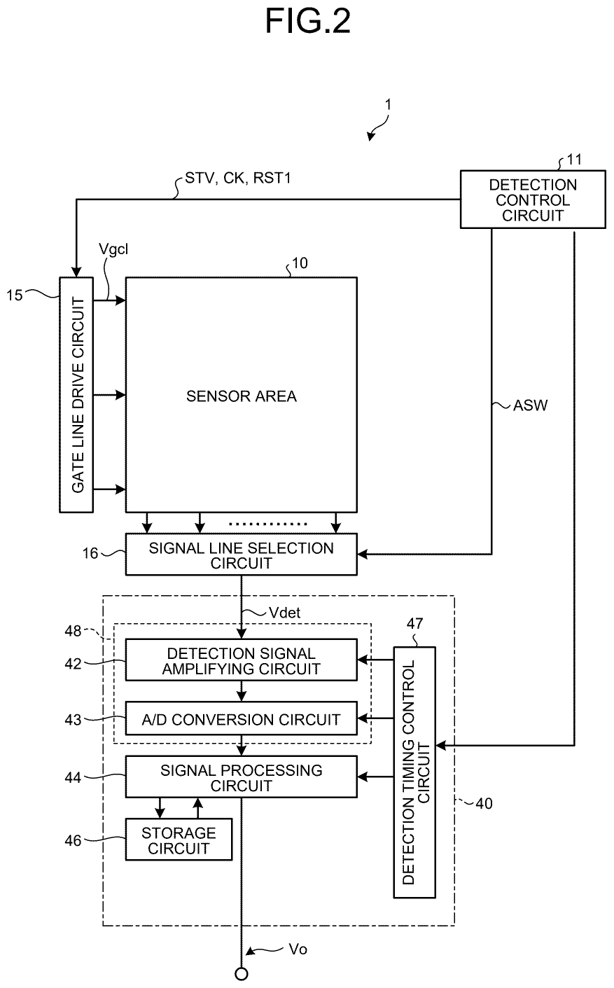

FIG. 2 is a block diagram illustrating a configuration example of the detection device according to the embodiment. As illustrated in FIG. 2, the detection device 1 further includes a detection control circuit 11 and a detection circuit 40.

The sensor area 10 includes the optical sensors PD. Each of the optical sensors PD included in the sensor area 10 is an organic photodiode (OPD), and outputs an electrical signal corresponding to light emitted thereto as a detection signal Vdet to the signal line selection circuit 16. The sensor area 10 performs detection in response to a gate drive signal Vgcl supplied from the gate line drive circuit 15.

The detection control circuit 11 is a circuit that supplies respective control signals to the gate line drive circuit 15, the signal line selection circuit 16, and the detection circuit 40 to control operations of these circuits. The detection control circuit 11 supplies various control signals such as a start signal STV, a clock signal CK, and a reset signal RST1 to the gate line drive circuit 15. The detection control circuit 11 also supplies various control signals such as a selection signal ASW to the signal line selection circuit 16. The detection control circuit 11 also supplies various control signals to the first and the second light sources 61 and 62 to control the lighting and the non-lighting of each group of the first and the second light sources 61 and 62.

The gate line drive circuit 15 is a circuit that drives a plurality of gate lines GCL (refer to FIG. 3) based on various control signals. The gate line drive circuit 15 sequentially or simultaneously selects the gate lines GCL and supplies the gate drive signal Vgcl to the selected gate lines GCL. Through this operation, the gate line drive circuit 15 selects the optical sensors PD coupled to the gate lines GCL.

The signal line selection circuit 16 is a switch circuit that sequentially or simultaneously selects a plurality of signal lines SGL (refer to FIG. 3). The signal line selection circuit 16 is a multiplexer, for example. The signal line selection circuit 16 electrically couples the selected signal lines SGL to the AFE circuit 48 based on selection signals ASW supplied from the detection control circuit 11. Through this operation, the signal line selection circuit 16 outputs the detection signals Vdet of the optical sensors PD to the detection circuit 40.

The detection circuit 40 includes the AFE circuit 48, a signal processing circuit 44, a storage circuit 46, and a detection timing control circuit 47. The detection timing control circuit 47 controls the AFE circuit 48 and the signal processing circuit 44 such that they operate in synchronization with each other, based on the control signal supplied from the detection control circuit 11.

The AFE circuit 48 detects the detection signals of the optical sensors PD output from the sensor area 10 in chronological order. The AFE circuit 48 is an analog front-end IC, for example.

The AFE circuit 48 is a signal processing circuit having functions of at least a detection signal amplifying circuit 42 and an analog-to-digital (A/D) conversion circuit 43. The detection signal amplifying circuit 42 amplifies the detection signals Vdet. The A/D conversion circuit 43 converts the analog signals output from the detection signal amplifying circuit 42 into digital signals at a predetermined sampling interval.

In the present disclosure, the control circuit 122 includes the signal processing circuit 44 and the storage circuit 46.

The signal processing circuit 44 acquires the biometric data for generating the information on the living body based on the detection values of the optical sensors PD output from the AFE circuit 48. In the present disclosure, the information on the living body includes the pulse waves acquired using the infrared light and/or red light.

The storage circuit 46 temporarily stores therein the signals processed by the signal processing circuit 44. In the present disclosure, the storage circuit 46 stores therein a biometric data acquisition area that is set in a biometric data acquisition area setting process flow (to be described later) when the signal processing circuit 44 acquires the biometric data, and also stores therein various types of setting information. The storage circuit 46 may have a configuration including a random-access memory (RAM), a read-only memory (ROM), an electrically erasable programmable read-only memory (EEPROM), and the like. The storage circuit 46 may be a register circuit or the like.

The following describes a circuit configuration example of the detection device 1. FIG. 3 is a circuit diagram illustrating the detection device according to the embodiment. As illustrated in FIG. 3, the sensor area 10 has a plurality of partial detection areas PAA arranged in a matrix having a row-column configuration. Each of the partial detection areas PAA is provided with the optical sensor PD.

The gate lines GCL extend in the first direction Dx and are each coupled to the partial detection areas PAA arranged in the first direction Dx. A plurality of gate lines GCL(1), GCL(2), . . . , GCL(8) are arranged in the second direction Dy and are each coupled to the gate line drive circuit 15. In the following description, the gate lines GCL(1), GCL(2), . . . , GCL(8) will each be simply referred to as the gate line GCL when they need not be distinguished from one another. To facilitate understanding of the description, FIG. 3 illustrates eight gate lines GCL. However, this is merely an example, and M gate lines GCL may be arranged (where M is a natural number, such as 256).

The signal lines SGL extend in the second direction Dy and are each coupled to the optical sensors PD of the partial detection areas PAA arranged in the second direction Dy. A plurality of signal lines SGL(1), SGL(2), . . . , SGL(12) are arranged in the first direction Dx and are each coupled to the signal line selection circuit 16 and a reset circuit 17. In the following description, the signal lines SGL(1), SGL(2), . . . , SGL(12) will each be simply referred to as the signal line SGL when they need not be distinguished from one another.

To facilitate understanding of the description, 12 signal lines SGL are illustrated. However, this is merely an example, and N signal lines SGL may be arranged (where N is a natural number, such as 252). In FIG. 3, the sensor area 10 is provided between the signal line selection circuit 16 and the reset circuit 17. The signal line selection circuit 16 and the reset circuit 17 are not limited to being provided in this way and may be coupled to ends of the signal lines SGL in the same direction.

The gate line drive circuit 15 receives the various control signals such as the start signal STV, the clock signal CK, and the reset signal RST1 from the control circuit 122 (refer to FIG. 1). The gate line drive circuit 15 sequentially selects the gate lines GCL(1), GCL(2), . . . , GCL(8) in a time-division manner based on the various control signals. The gate line drive circuit 15 supplies the gate drive signal Vgcl to the selected one of the gate lines GCL. This operation supplies the gate drive signal Vgcl to a plurality of first switching elements Tr coupled to the gate line GCL and thus selects the partial detection areas PAA arranged in the first direction Dx as detection targets.

The gate line drive circuit 15 may perform different driving for each of detection modes including the detection of the fingerprint and the detection of a plurality of different items of information on a living body (including, for example, the pulse waves, the pulsation, the vascular image, and the blood oxygen level, which are hereinafter called also simply “biometric information”). For example, the gate line drive circuit 15 may collectively drive more than one of the gate lines GCL.

Specifically, the gate line drive circuit 15 simultaneously selects a predetermined number of the gate lines GCL from among the gate lines GCL(1), GCL(2), . . . , GCL(8) based on the control signals. For example, the gate line drive circuit 15 simultaneously selects six gate lines GCL(1) to GCL(6) and supplies thereto the gate drive signals Vgcl. The gate line drive circuit 15 supplies the gate drive signals Vgcl via the selected six gate lines GCL to the first switching elements Tr. This operation selects block units PAG1 and PAG2 each including more than one of the partial detection areas PAA arranged in the first direction Dx and the second direction Dy as the detection targets. The gate line drive circuit 15 collectively drives the predetermined number of the gate lines GCL and sequentially supplies the gate drive signals Vgcl to each unit of the predetermined number of the gate lines GCL.

The signal line selection circuit 16 includes a plurality of selection signal lines Lsel, a plurality of output signal lines Lout, and third switching elements TrS. The third switching elements TrS are provided correspondingly to the respective signal lines SGL. Six signal lines SGL(1), SGL(2), . . . , SGL(6) are coupled to the same output signal line Lout1. Six signal lines SGL(7), SGL(8), . . . , SGL(12) are coupled to the same output signal line Lout2. The output signal lines Lout1 and Lout2 are each coupled to the AFE circuit 48.

The signal lines SGL(1), SGL(2), . . . , SGL(6) are grouped into a first signal line block, and the signal lines SGL(7), SGL(8), . . . , SGL(12) are grouped into a second signal line block. The selection signal lines Lsel are coupled to the gates of the respective third switching elements TrS included in one of the signal line blocks. One of the selection signal lines Lsel is coupled to the gates of the third switching elements TrS in the signal line blocks.

Specifically, selection signal lines Lsel1, Lsel2, . . . , Lsel6 are coupled to the third switching elements TrS corresponding to the signal lines SGL(1), SGL(2), . . . , SGL(6), respectively. The selection signal line Lsel1 is coupled to the third switching element TrS corresponding to the signal line SGL(1) and the third switching element TrS corresponding to the signal line SGL(7). The selection signal line Lsel2 is coupled to the third switching element TrS corresponding to the signal line SGL(2) and the third switching element TrS corresponding to the signal line SGL(8).

The control circuit 122 (refer to FIG. 1) sequentially supplies the selection signal ASW to the selection signal lines Lsel. This operation causes the signal line selection circuit 16 to operate the third switching elements TrS to sequentially select the signal lines SGL in one of the signal line blocks in a time-division manner. The signal line selection circuit 16 selects one of the signal lines SGL in each of the signal line blocks. Such a configuration can reduce the number of integrated circuits (ICs) including the AFE circuit 48 or the number of terminals of the ICs in the detection device 1.

The signal line selection circuit 16 may collectively couple more than one of the signal lines SGL to the AFE circuit 48. Specifically, the control circuit 122 (refer to FIG. 1) simultaneously supplies the selection signal ASW to the selection signal lines Lsel. The signal line selection circuit 16 operates the third switching elements TrS to select the signal lines SGL (for example, six signal lines SGL) in one of the signal line blocks, and couples the signal lines SGL to the AFE circuit 48. As a result, signals detected in each of the block units PAG1 and PAG2 are output to the AFE circuit 48. In this case, the signals from the partial detection areas PAA (optical sensors PD) included in each of the block units PAG1 and PAG2 are integrated and output to the AFE circuit 48.

The detection is performed for each of the block units PAG1 and PAG2 by the operations of the gate line drive circuit 15 and the signal line selection circuit 16. As a result, the intensity of the detection signal Vdet obtained by a one-time detection operation increases, so that the sensor sensitivity can be improved.

In the present disclosure, the detection device 1 can change the number of the partial detection areas PAA (optical sensors PD) included in each of the block units PAG1 and PAG2. Thus, the value of resolution per inch (pixels per inch (ppi), hereinafter, referred to as “definition”) can be set depending on the information to be acquired.

For example, the number of the partial detection areas PAA (optical sensors PD) included in each of the block units PAG1 and PAG2 is relatively reduced. While this setting results in a longer detection time and a lower frame rate (for example, 20 frames per second (fps) or lower), the detection can be performed at a higher definition (for example, at 300 ppi or higher). Hereafter, the term “first mode” denotes a mode of performing the detection at a lower frame rate and a higher definition. By selecting the first mode of performing the detection at a lower frame rate and a higher definition, for example, the fingerprint on the surface of a finger can be acquired at a higher definition.

Alternatively, for example, the number of the partial detection areas PAA (optical sensors PD) included in each of the block units PAG1 and PAG2 is relatively increased. While this setting results in a lower definition (for example, 50 ppi or lower), the detection can be performed at a higher frame rate (for example, at 100 fps or higher) that allows the detection to be repeatedly performed in a shorter time in one frame. Hereafter, the term “second mode” denotes a mode of performing the detection at a higher frame rate and a lower definition. By selecting the second mode of performing the detection at a higher frame rate and a lower definition, for example, a change in pulse wave with time can be more accurately detected. In the second mode, calculation of a pulse wave velocity, calculation of blood pressure, and the like are enabled by using the pulse waves acquired at a higher frame rate (for example, 1000 fps or higher).

For example, when acquiring the vascular image (vein pattern), the number of the partial detection areas PAA (optical sensors PD) included in each of the block units PAG1 and PAG2 is set to an intermediate value between those of the first mode and the second mode. This setting allows the detection to be performed at a medium frame rate that is higher than that of the first mode and lower than that of the second mode (for example, higher than 20 fps and lower than 100 fps) and at a medium definition that is lower than that of the first mode and higher than that of the second mode (for example, higher than 50 ppi and lower than 300 ppi). Hereafter, the term “third mode” denotes a mode of performing the detection at the medium frame rate and the medium definition. The third mode of performing the detection at the medium frame rate and the medium definition is suitable, for example, for acquiring the vascular pattern of veins and the like.

As illustrated in FIG. 3, the reset circuit 17 includes a reference signal line Lvr, a reset signal line Lrst, and fourth switching elements TrR. The fourth switching elements TrR are provided correspondingly to the signal lines SGL. The reference signal line Lvr is coupled to either the sources or the drains of the fourth switching elements TrR. The reset signal line Lrst is coupled to the gates of the fourth switching elements TrR.

The control circuit 122 supplies a reset signal RST2 to the reset signal line Lrst. This operation turns on the fourth switching elements TrR to electrically couple the signal lines SGL to the reference signal line Lvr. The power supply circuit 123 supplies a reference signal COM to the reference signal line Lvr. This operation supplies the reference signal COM to a capacitive element Ca (refer to FIG. 4) included in each of the partial detection areas PAA.

FIG. 4 is a circuit diagram illustrating the partial detection areas of the detection device according to the embodiment. FIG. 4 also illustrates a circuit configuration of the AFE circuit 48. As illustrated in FIG. 4, each of the partial detection areas PAA includes the optical sensor PD, the capacitive element Ca, and a corresponding one of the first switching elements Tr. The capacitive element Ca is capacitance (sensor capacitance) generated in the optical sensor PD and is equivalently coupled in parallel to the optical sensor PD. In addition, signal line capacitance Cc is parasitic capacitance generated on the signal line SGL and is equivalently provided between the signal line SGL and both the anode of the optical sensor PD and one end side of the capacitive element Ca.

FIG. 4 illustrates two gate lines GCL(m) and GCL(m+1) arranged in the second direction Dy among the gate lines GCL. FIG. 4 also illustrates two signal lines SGL(n) and SGL(n+1) arranged in the first direction Dx among the signal lines SGL. The partial detection area PAA is an area surrounded by the gate lines GCL and the signal lines SGL.

Each of the first switching elements Tr is provided correspondingly to the optical sensor PD. The first switching element Tr is made of a thin-film transistor, and in this example, made of an n-channel metal oxide semiconductor (MOS) thin-film transistor (TFT).

The gates of the first switching elements Tr belonging to the partial detection areas PAA arranged in the first direction Dx are coupled to the gate line GCL. The sources of the first switching elements Tr belonging to the partial detection areas PAA arranged in the second direction Dy are coupled to the signal line SGL. The drain of the first switching element Tr is coupled to the cathode of the optical sensor PD and the capacitive element Ca.

The anode of the optical sensor PD is supplied with the sensor power supply signal (potential) VDDSNS from the power supply circuit 123. The cathode of the optical sensor PD is supplied with the reference signal COM that serves as an initial potential of the signal line SGL and the capacitive element Ca from the power supply circuit 123.

When the partial detection area PAA is irradiated with light, a current corresponding to an amount of light flows through the optical sensor PD. As a result, an electric charge corresponding to the amount of light is stored in the capacitive element Ca. Turning on the first switching element Tr causes a current corresponding to the electric charge stored in the capacitive element Ca to flow through the signal line SGL. The signal line SGL is coupled to the AFE circuit 48 through a corresponding one of the third switching elements TrS of the signal line selection circuit 16. Thus, the detection device 1 can detect a signal corresponding to the amount of light irradiating the optical sensor PD in each of the partial detection areas PAA or signals corresponding to the amounts of light irradiating the optical sensors PD in each of the block units PAG1 and PAG2.

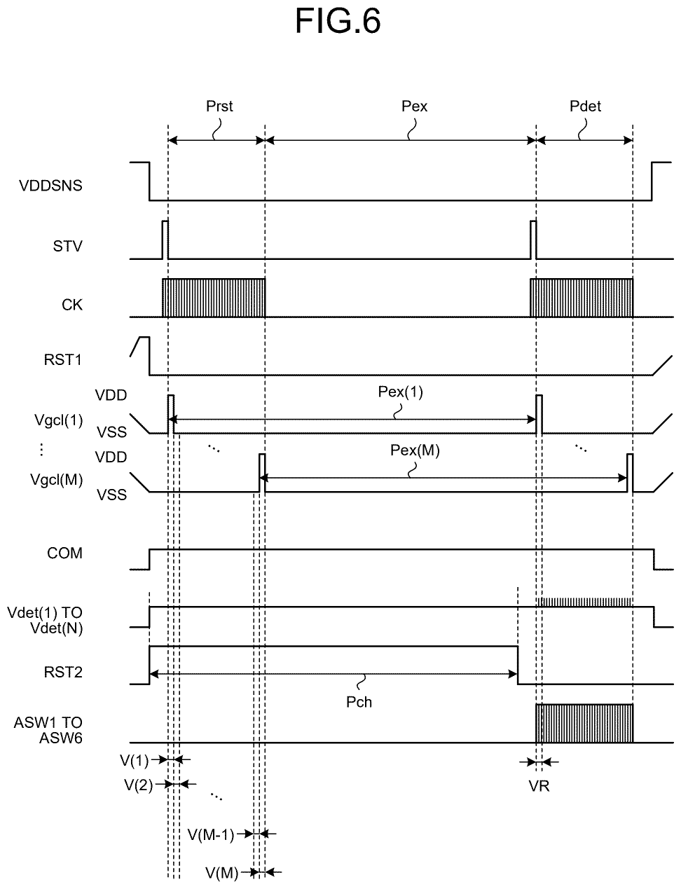

During a readout period Pdet (refer to FIG. 6), a switch SSW of the AFE circuit 48 is turned on to couple the AFE circuit 48 to the signal line SGL. The detection signal amplifying circuit 42 of the AFE circuit 48 converts a current supplied from the signal lines SGL into a voltage, and amplifies the result. A reference potential (Vref) having a fixed potential is supplied to a non-inverting input part (+) of the detection signal amplifying circuit 42, and the signal lines SGL are coupled to an inverting input terminal (−) of the detection signal amplifying circuit 42. In the embodiment, the same signal as the reference signal COM is supplied as the reference potential (Vref) voltage. The detection signal amplifying circuit 42 includes a capacitive element Cb and a reset switch RSW. During a reset period Prst (refer to FIG. 6), the reset switch RSW is turned on, and the electric charge of the capacitive element Cb is reset.

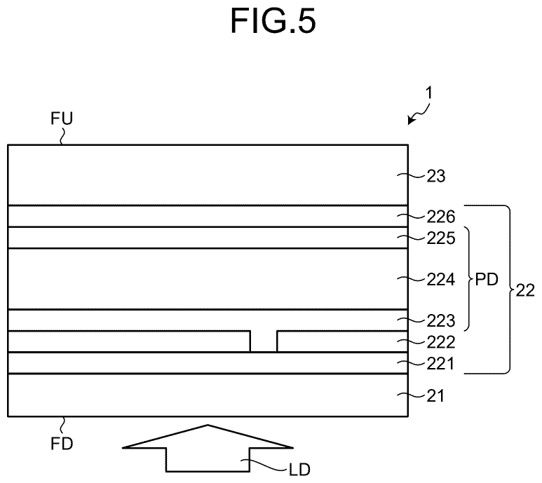

The following describes a configuration of the optical sensor PD. FIG. 5 is a schematic partial sectional view of the optical sensor according to the embodiment. The sensor area 10 of the detection device 1 includes the sensor base member 21, a sensor structure 22, and a protective film 23. The sensor base member 21 is, for example, an insulating base member formed of a film-like resin.

The sensor structure 22 includes a TFT layer 221, an anode electrode (lower electrode) 222, the optical sensor PD, and a cathode electrode (upper electrode) 226.

The TFT layer 221 is provided with various types of wiring such as the gate lines GCL and the signal lines SGL. The sensor base member 21 and the TFT layer 221 are a drive circuit that drives the sensor and are also called a backplane.

The optical sensor PD includes an active layer 224, an electron transport layer (lower buffer layer) 223, and a hole transport layer (upper buffer layer) 225. The electron transport layer 223 is provided between the active layer 224 and the anode electrode (lower electrode) 222. The hole transport layer 225 is provided between the active layer 224 and the cathode electrode (upper electrode) 226. In other words, the electron transport layer (lower buffer layer) 223, the active layer 224, and the hole transport layer (upper buffer layer) 225 of the optical sensor PD are stacked in this order in a direction orthogonal to the sensor base member 21.

The active layer 224 changes in characteristics (for example, voltage-current characteristics and resistance value) according to light emitted thereto. An organic material is used as a material of the active layer 224. Specifically, the active layer 224 has a bulk heterostructure containing a mixture of a p-type organic semiconductor and an n-type fullerene derivative ((6,6)-phenyl-C61-butyric acid methyl ester (PCBM)) that is an n-type organic semiconductor. As the active layer 224, low-molecular-weight organic materials can be used including, for example, fullerene (C60), phenyl-C61-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 224 can be formed by a vapor deposition process (dry process) using any of the low-molecular-weight organic materials listed above. In this case, the active layer 224 may be, for example, a multilayered film of CuPc and F₁₆CuPc, or a multilayered film of rubrene and C₆₀. The active layer 224 can also be formed by a coating process (wet process). In this case, the active layer 224 is made using a material obtained by combining any of the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The active layer 224 can be a film made of a mixture of P3HT and PCBM, or a film made of a mixture of F8BT and PDI.

The electron transport layer (lower buffer layer) 223 and the hole transport layer (upper buffer layer) 225 are provided to facilitate electrons and holes generated in the active layer 224 to reach the anode electrode (lower electrode) 222 or the cathode electrode (upper electrode) 226. The electron transport layer (lower buffer layer) 223 is in direct contact with the top of the anode electrode (lower electrode) 222. The active layer 224 is in direct contact with the top of the electron transport layer (lower buffer layer) 223. Polyethylenimine ethoxylated (PEIE) is used as a material of the electron transport layer (lower buffer layer) 223.

The hole transport layer (upper buffer layer) 225 is in direct contact with the top of the active layer 224, and the cathode electrode (upper electrode) 226 is in direct contact with the top of the hole transport layer (upper buffer layer) 225. The hole transport layer (upper buffer layer) 225 is a metal oxide layer. For example, tungsten oxide (WO₃) or molybdenum oxide is used as the metal oxide layer.

The materials and the manufacturing methods of the electron transport layer (lower buffer layer) 223, the active layer 224, and the hole transport layer (upper buffer layer) 225 are merely exemplary, and other materials and manufacturing methods may be used.

The anode electrode (lower electrode) 222 faces the cathode electrode (upper electrode) 226 with the optical sensor PD interposed therebetween. A light-transmitting conductive material such as indium tin oxide (ITO) is used as the cathode electrode (upper electrode) 226. A metal material such as silver (Ag) or aluminum (Al) is used as the anode electrode (lower electrode) 222. Alternatively, the anode electrode (lower electrode) 222 may be an alloy material containing at least one or more of these metal materials.

The anode electrode (lower electrode) 222 can be formed as a light-transmitting transflective electrode by controlling the film thickness of the anode electrode (lower electrode) 222. For example, the anode electrode (lower electrode) 222 is formed of an Ag thin film having a thickness of 10 nm so as to have light transmittance of approximately 60%. In this case, the optical sensor PD can detect, for example, first light LD entering from a first surface FD side.

The protective film 23 is provided on a second surface FU so as to cover the cathode electrode (upper electrode) 226. The protective film 23 is a passivation film and is provided to protect the optical sensor PD.

As illustrated in FIG. 4, the sensor power supply signal VDDSNS is supplied from the power supply circuit 123 to the anode of the optical sensor PD, and the reference signal COM serving as the initial potential of both the signal line SGL and the capacitive element Ca is supplied from the power supply circuit 123 to the cathode of the optical sensor PD. However, for example, a configuration may be employed in which: the sensor power supply signal VDDSNS is supplied from the power supply circuit 123 to the cathode of the optical sensor PD; and the reference signal COM serving as the initial potential of both the signal line SGL and the capacitive element Ca is supplied from the power supply circuit 123 to the anode of the optical sensor PD. In this case, unlike in the configuration described above, the optical sensor PD includes the active layer 224, the hole transport layer (lower buffer layer) 223, and the electron transport layer (upper buffer layer) 225. In this case, the hole transport layer (lower buffer layer) 223 is provided between the active layer 224 and the cathode electrode (lower electrode) 222, and the electron transport layer (upper buffer layer) 225 is provided between the active layer 224 and the anode electrode (upper electrode) 226. In other words, the hole transport layer (lower buffer layer) 223, the active layer 224, and the electron transport layer (upper buffer layer) 225 of the optical sensor PD are stacked in this order in the direction orthogonal to the sensor base member 21.

In the present disclosure, the optical sensor PD is not limited to an organic photodiode (OPD). The optical sensor PD may be a silicon photodiode (SiPD), for example.

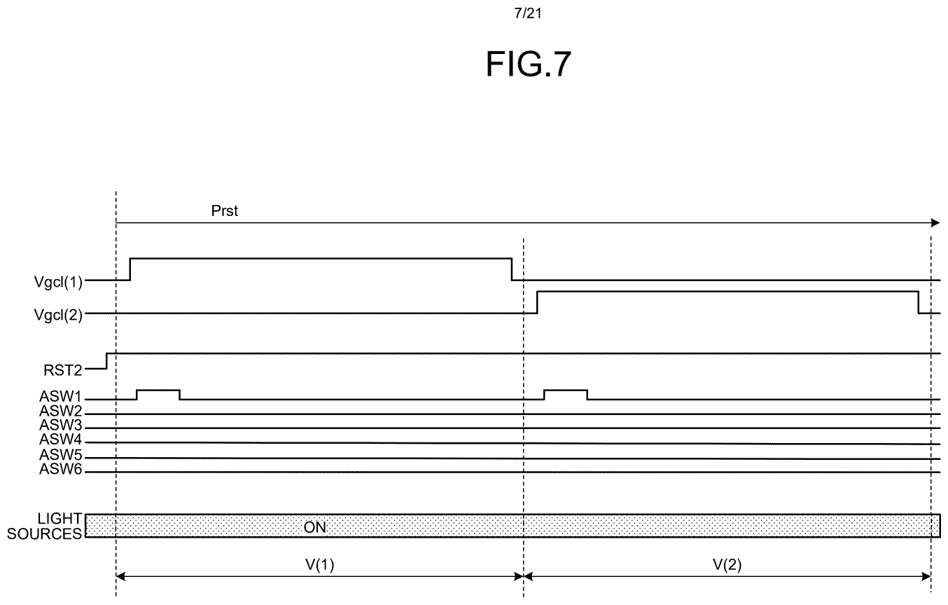

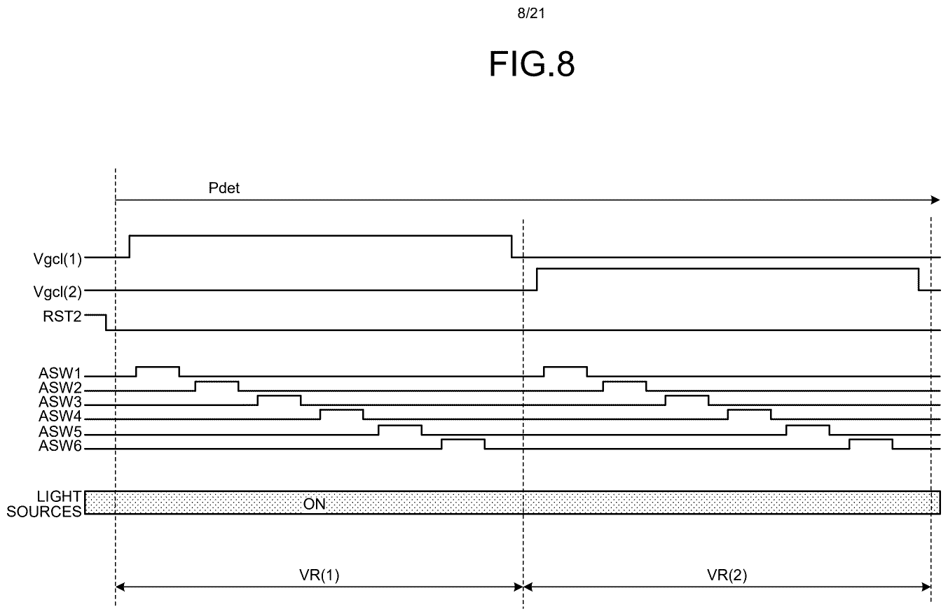

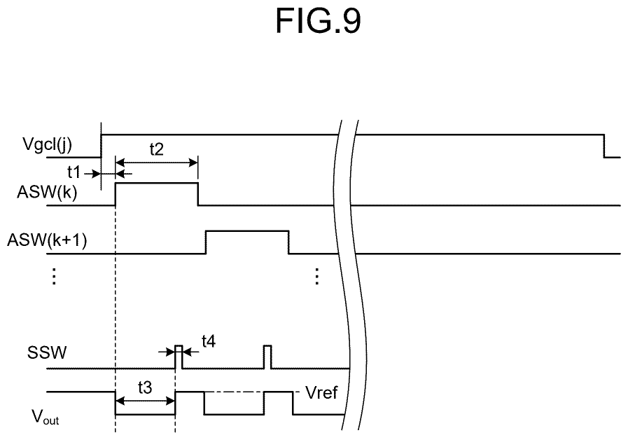

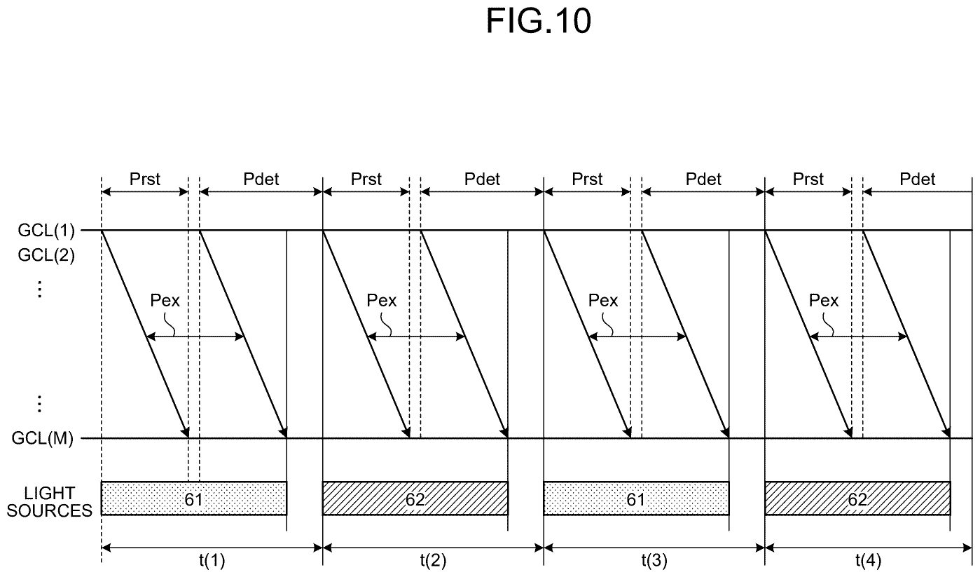

The following describes an operation example of the detection device 1. FIG. 6 is a timing waveform diagram illustrating the operation example of the detection device according to the embodiment. FIG. 7 is a timing waveform diagram illustrating an operation example in the reset period in FIG. 6. FIG. 8 is a timing waveform diagram illustrating an operation example in the readout period in FIG. 6. FIG. 9 is a timing waveform diagram illustrating an operation example in a drive period for one gate line included in the readout period VR in FIG. 6. FIG. 10 is an explanatory diagram for explaining a first example of a relation between driving of the sensor area and lighting operations of the light sources in the detection device according to the embodiment.

As illustrated in FIG. 6, the detection device 1 has the reset period Prst, an exposure period Pex, and the readout period Pdet. The power supply circuit 123 supplies the sensor power supply signal VDDSNS to the anode of the optical sensor PD over the reset period Prst, the exposure period Pex, and the readout period Pdet. The sensor power supply signal VDDSNS is a signal that applies a reverse bias between the anode and the cathode of the optical sensor PD. For example, the reference signal COM of substantially 0.75 V is applied to the cathode of the optical sensor PD, and the sensor power supply signal VDDSNS of substantially-1.25 V is applied to the anode thereof. As a result, a reverse bias of substantially 2.0 V is applied between the anode and the cathode. The control circuit 122 sets the reset signal RST2 to “H”, and then, supplies the start signal STV and the clock signal CK to the gate line drive circuit 15 to start the reset period Prst. During the reset period Prst, the control circuit 122 supplies the reference signal COM to the reset circuit 17 and uses the reset signal RST2 to turn on the fourth switching elements TrR for supplying a reset voltage. This operation supplies the reference signal COM as the reset voltage to each of the signal lines SGL. The reference signal COM is set to 0.75 V, for example.

During the reset period Prst, the gate line drive circuit 15 sequentially selects the gate lines GCL based on the start signal STV, the clock signal CK, and the reset signal RST1. The gate line drive circuit 15 sequentially supplies gate drive signals Vgcl {Vgcl(1), . . . , Vgcl(M)} to the gate lines GCL. Each of the gate drive signals Vgcl has a pulsed waveform having a power supply voltage VDD serving as a high-level voltage and a power supply voltage VSS serving as a low-level voltage. In FIG. 6, M gate lines GCL are provided (where M is, for example, 256), and the gate drive signals Vgcl(1), . . . , Vgcl(M) are sequentially supplied to the respective gate lines GCL. Thus, the first switching elements Tr are sequentially brought into a conducting state and supplied with the reset voltage on a row-by-row basis. For example, a voltage of 0.75 V of the reference signal COM is supplied as the reset voltage.

Specifically, as illustrated in FIG. 7, the gate line drive circuit 15 supplies the gate drive signal Vgcl(1) at the high-level voltage (power supply voltage VDD) to the gate line GCL(1) during a period V(1). The control circuit 122 supplies at least one of selection signals ASW1, . . . , ASW6 (selection signal ASW1 in FIG. 7) to the signal line selection circuit 16 during a period in which the gate drive signal Vgcl(1) is at the high-level voltage (power supply voltage VDD). This operation couples the signal line SGL of the partial detection area PAA selected by the selection signal ASW1 to the AFE circuit 48. As a result, the reset voltage (reference signal COM) is also supplied to coupling wiring between the third switching element Trs and the AFE circuit 48.

In the same way, the gate line drive circuit 15 supplies the gate drive signals Vgcl(2), . . . , Vgcl(M−1), Vgcl(M) at the high-level voltage to gate lines GCL(2), . . . , GCL(M−1), GCL(M) during periods V(2), . . . , V(M−1), V(M), respectively.

Thus, during the reset period Prst, the capacitive elements Ca of all the partial detection areas PAA are sequentially electrically coupled to the signal lines SGL and are supplied with the reference signal COM. As a result, the capacitance of the capacitive elements Ca is reset. The capacitance of the capacitive elements Ca of some of the partial detection areas PAA can be reset by selecting some of the gate lines and some of the signal lines SGL.

Examples of the method of controlling the exposure include a method of controlling the exposure during non-selection of the gate lines and a method of always controlling the exposure. In the method of controlling the exposure during non-selection of the gate lines, the gate drive signals {Vgcl(1), . . . , Vgcl(M)} are sequentially supplied to all the gate lines GCL coupled to the optical sensors PD serving as the detection targets, and all the optical sensors PD serving as the detection targets are supplied with the reset voltage. Then, after all the gate lines GCL coupled to the optical sensors PD serving as the detection targets are set to a low voltage (the first switching elements Tr are turned off), the exposure starts and the exposure is performed during the exposure period Pex. After the exposure ends, the gate drive signals {Vgcl(1), . . . , Vgcl(M)} are sequentially supplied to the gate lines GCL coupled to the optical sensors PD serving as the detection targets as described above, and reading is performed during the readout period Pdet. In the method of always controlling the exposure, the control for performing the exposure can also be performed during the reset period Prst and the readout period Pdet (the exposure is always controlled). In this case, the exposure period Pex(1) starts after the gate drive signal Vgcl(1) is supplied to the gate line GCL during the reset period Prst. The exposure periods Pex {(1), . . . , (M)} are substantial exposure periods during which the capacitive elements Ca are charged from the optical sensors PD. No light is emitted except in these periods. The electric charge stored in the capacitive element Ca during the reset period Prst causes a reverse-directional current (from cathode to anode) to flow through the optical sensor PD due to light irradiation, and the potential difference between both ends of the capacitive element Ca decreases. The start timing and the end timing of the substantial exposure periods Pex(1), . . . , Pex(M) are different among the partial detection areas PAA corresponding to the gate lines GCL. Each of the exposure periods Pex(1), . . . , Pex(M) starts when the gate drive signal Vgcl changes from the power supply voltage VDD serving as the high-level voltage to the power supply voltage VSS serving as the low-level voltage during the reset period Prst. Each of the exposure periods Pex(1), . . . , Pex(M) ends when the gate drive signal Vgcl changes from the power supply voltage VSS to the power supply voltage VDD during the readout period Pdet. The lengths of exposure time of the exposure periods Pex(1), . . . , Pex(M) are equal.

During the exposure periods Pex {(1) . . . (M)}, current flows correspondingly to the light received by the optical sensor PD in each of the partial detection areas PAA. As a result, an electric charge is stored in each of the capacitive elements Ca.

At a time before the readout period Pdet starts, the control circuit 122 sets the reset signal RST2 to a low-level voltage. This operation stops the operation of the reset circuit 17. The reset signal may be set to a high-level voltage only during the reset period Prst. During the readout period Pdet, the gate line drive circuit 15 sequentially supplies the gate drive signals Vgcl(1), . . . , Vgcl(M) to the gate lines GCL in the same way as during the reset period Prst.

Specifically, as illustrated in FIG. 8, the gate line drive circuit 15 supplies the gate drive signal Vgcl(1) at the high-level voltage (power supply voltage VDD) to the gate line GCL(1) during a row readout period VR(1). The control circuit 122 sequentially supplies the selection signals ASW1, ASW6 to the signal line selection circuit 16 while the gate drive signal Vgcl(1) is at the high-level voltage (power supply voltage VDD). This operation sequentially couples the signal lines SGL of the partial detection areas PAA selected by the gate drive signal Vgcl(1) to the AFE circuit 48. As a result, the detection signal Vdet of each of the partial detection areas PAA is supplied to the AFE circuit 48. A predetermined number of signals among the selection signals ASW1, . . . , ASW6 may be simultaneously supplied to the signal line selection circuit 16. In this case, the predetermined number of the signal lines SGL of the partial detection areas PAA selected by the gate drive signal Vgcl(1) are coupled to the AFE circuit 48.

In the same way, the gate line drive circuit 15 supplies the gate drive signals Vgcl(2), . . . , Vgcl(M−1), Vgcl(M) at the high-level voltage to the gate lines GCL(2), . . . . GCL(M−1), GCL(M) during row readout periods VR(2), . . . , VR(M−1), VR(M), respectively. That is, the gate line drive circuit 15 supplies the gate drive signal Vgcl to the gate line GCL during each of the row readout periods VR(1), VR(2), . . . . VR(M−1), VR(M). The signal line selection circuit 16 sequentially or simultaneously selects the signal lines SGL based on the selection signal ASW during each period in which the gate drive signal Vgcl is set to the high-level voltage. The signal line selection circuit 16 sequentially or simultaneously couples each of the signal lines SGL to one AFE circuit 48. Thus, the detection device 1 can output the detection signals Vdet of all the partial detection areas PAA to the AFE circuit 48 during the readout period Pdet.

With reference to FIG. 9, the following describes an operation example during the row readout period VR that is a supply period of one gate drive signal Vgcl(j) in FIG. 6. In FIG. 6, the reference numeral of the row readout period VR is assigned to the first gate drive signal Vgcl(1). The same also applies to the other gate drive signals Vgcl(2), . . . , Vgcl(M). j is any one of the natural numbers 1 to M.

As illustrated in FIGS. 9 and 4, an output (Vout) of each of the third switching elements TrS has been reset to the reference potential (Vref) voltage in advance. The reference potential (Vref) voltage serves as the reset voltage, and is set to 0.75 V, for example. Then, the gate drive signal Vgcl(j) is set to a high level, and the first switching elements Tr of a corresponding row are turned on. Thus, each of the signal lines SGL in each row is set to a voltage corresponding to the electric charge stored in the capacitance (capacitive element Ca) of the partial detection area PAA. After a period t1 elapses from a rising edge of the gate drive signal Vgcl(j), a period t2 starts in which the selection signal ASW(k) is set to a high level. After the selection signal ASW(k) is set to the high level and the third switching element TrS is turned on, the AFE circuit 48 is electrically coupled to the capacitance (capacitive element Ca) of the partial detection area PAA via the third switching element TrS. This operation changes the output (Vout) of the third switching element TrS (refer to FIG. 4) to a voltage corresponding to the electric charge stored in the capacitance (capacitive element Ca) of the partial detection area PAA (period t3). In the example of FIG. 9, this voltage is reduced from the reset voltage as illustrated in the period t3. Then, after the switch SSW is turned on (period t4 during which an SSW signal is set to a high level), the electric charge stored in the capacitance (capacitive element Ca) of the partial detection area PAA is transferred to the capacitance (capacitive element Cb) of the detection signal amplifying circuit 42 of the AFE circuit 48, and the output voltage of the detection signal amplifying circuit 42 is set to a voltage corresponding to the electric charge stored in the capacitive element Cb. At this time, the potential of the inverting input part of the detection signal amplifying circuit 42 is set to a virtual short-circuit potential of an operational amplifier, and therefore, set to the reference potential (Vref). The A/D conversion circuit 43 reads out the output voltage of the detection signal amplifying circuit 42. In the example of FIG. 9, the waveforms of the selection signals ASW(k), ASW(k+1), . . . corresponding to the signal lines SGL of the respective columns are set high to sequentially turn on the third switching elements TrS, and the same operation is sequentially performed to sequentially read out the electric charges stored in the capacitance (capacitive elements Ca) of the partial detection areas PAA coupled to the gate line GCL. ASW(k), ASW(k+1), . . . in FIG. 9 are, for example, any of ASW1 to ASW6 in FIG. 8.

Specifically, after the period t4 of turning on the switch SSW starts, the electric charge is transferred from the capacitance (capacitive element Ca) of the partial detection area PAA to the capacitance (capacitive element Cb) of the detection signal amplifying circuit 42 of the AFE circuit 48. At this time, the non-inverting input (+) of the detection signal amplifying circuit 42 is set to the reference potential (Vref) voltage (at 0.75 V, for example). Therefore, the output (Vout) of the third switching element TrS is also set to the reference potential (Vref) voltage due to the virtual short-circuit between the inputs of the detection signal amplifying circuit 42. The voltage of the capacitive element Cb is set to a voltage corresponding to the electric charge stored in the capacitance (capacitive element Ca) of the partial detection area PAA at a location where the third switching element TrS is turned on in response to the selection signal ASW(k). After the output (Vout) of the third switching element TrS is set to the reference potential (Vref) voltage due to the virtual short-circuit, the output of the detection signal amplifying circuit 42 reaches a voltage corresponding to the capacitance of the capacitive element Cb. The A/D conversion circuit 43 reads the output voltage of the detection signal amplifying circuit 42. The voltage of the capacitive element Cb is, for example, a voltage between two electrodes provided on a capacitor constituting the capacitive element Cb.

In a first example illustrated in FIG. 10, the detection device 1 executes the reset period Prst, the exposure periods Pex {(1), . . . , (M)}, and the readout period Pdet described above in each of the periods t(1), t(2), t(3), and t(4). In the reset period Prst and the readout period Pdet, the gate line drive circuit 15 sequentially scans the gate lines from GCL(1) to GCL(M). In the following description, the term “one-frame detection” denotes the detection in the periods t(1), t(2), t(3), and t(4), that is, the detection in which the gate lines are scanned from GCL(1) to GCL(M) in the reset period Prst and the readout period Pdet and the detection signals Vdet are acquired from the signal lines SGL in the respective columns.

The control circuit 122 can control the lighting and the non-lighting of the light sources according to the detection target. FIG. 10 illustrates an example in which the first light sources 61 are on during the periods t(1) and t(3), and the second light sources 62 are on during the periods t(2) and t(4). That is, in the first example illustrated in FIG. 10, the control circuit 122 alternately switches the first light sources 61 and the second light sources 62 on and off for each one-frame detection. The present disclosure is not limited to this example. For example, the control circuit 122 may switch the first light sources 61 and the second light sources 62 on and off at intervals of a predetermined period of time, or may continuously turn on either of the first and the second light sources 61 and 62.

FIGS. 6 to 10 illustrate the example in which the gate line drive circuit 15 individually selects each of the gate lines GCL, but the present disclosure is not limited to this example. The gate line drive circuit 15 may simultaneously select a predetermined number (two or more) of the gate lines GCL, and sequentially supply the gate drive signals Vgcl to each unit of the predetermined number of the gate lines GCL. The signal line selection circuit 16 may also simultaneously couple a predetermined number (two or more) of the signal lines SGL to one AFE circuit 48. Moreover, the gate line drive circuit 15 may scan some of the gate lines GCL while skipping the others.

As illustrated in FIG. 8, in the row readout period VR(1), the selection signals ASW1, ASW6 are sequentially supplied to the signal line selection circuit 16 during the period in which the gate drive signal Vgcl(1) is at the high-level voltage (power supply voltage VDD). As described above, the detection device 1 has the configuration including, for example, a plurality of types of light sources (first light sources 61 and second light sources 62) that emit light having different wave lengths, and thereby, can acquire the fingerprint acquired by detecting the light reflected on the surface of the finger of the subject of examination and the various types of biometric information acquired by detecting the light reflected in or transmitted through the finger or the wrist of the subject of examination.

As a specific example of the information on the living body acquired by the detection device 1, the following describes an example of acquiring the pulse waves serving as the biometric information for calculating the oxygen saturation level in blood (hereinafter, called “blood oxygen saturation level (SpO₂)”).

When acquiring the pulse waves for calculating the blood oxygen saturation level (SpO₂), for example, the red visible light (red light) having a wavelength of 600 nm to 700 nm, specifically, approximately 660 nm, is employed as the first light emitted from the first light sources 61, and the infrared light having a wavelength of 780 nm to 950 nm, specifically, approximately 850 nm, is employed as the second light emitted from the second light sources 62. When acquiring the human blood oxygen saturation level (SpO₂), a pulse wave acquired using the first light (red light) and a pulse wave acquired using the second light (infrared light) are used.

Since the amount of light absorbed changes depending on the amount of oxygen taken in by hemoglobin, the optical sensor PD detects the amount of light obtained by subtracting the light absorbed by blood (hemoglobin) from the applied first and second light. Most of the oxygen in blood is reversibly bound to hemoglobin in red blood cells, and a small portion thereof is dissolved in blood plasma. More specifically, the value indicating what percentage of a permissible amount of oxygen is bound to blood as a whole is called the oxygen saturation level (SpO₂). At the two wavelengths of the first light and the second light, the blood oxygen saturation level can be calculated from the amount obtained by subtracting the light absorbed by blood (hemoglobin) from the applied light.

In the first example illustrated in FIG. 10, the reset period Prst, the exposure period Pex, and the readout period Pdet are provided in the detection for one frame in each of the periods t(1), t(2), t(3), and t(4). In the reset period Prst and the readout period Pdet, the gate line drive circuit 15 sequentially scans the gate lines from GCL(1) to GCL(M).

In the first example illustrated in FIG. 10, in the detection for one frame in the period t(1), the control circuit 122 (detection control circuit 11) turns on the first light sources 61 and turns off the second light sources 62 during the exposure period Pex. In the detection for one frame in the period t(2), the control circuit 122 (detection control circuit 11) turns off the first light sources 61 and turns on the second light sources 62 during the exposure period Pex. In the same way, the first light sources 61 are turned on and the second light sources 62 are turned off during the exposure period Pex in the detection for one frame in the period t(3), and the first light sources 61 are turned off and the second light sources 62 are turned on during the exposure period Pex in the detection for one frame in the period t(4).

Thus, in the first example illustrated in FIG. 10, the first and the second light sources 61 and 62 are controlled to be turned on and off in a time-division manner for each detection operation for one frame. As a result, a first detection value detected by the optical sensor PD based on the first light and a second detection value detected by the optical sensor PD based on the second light are output to the AFE circuit 48 in a time-division manner.

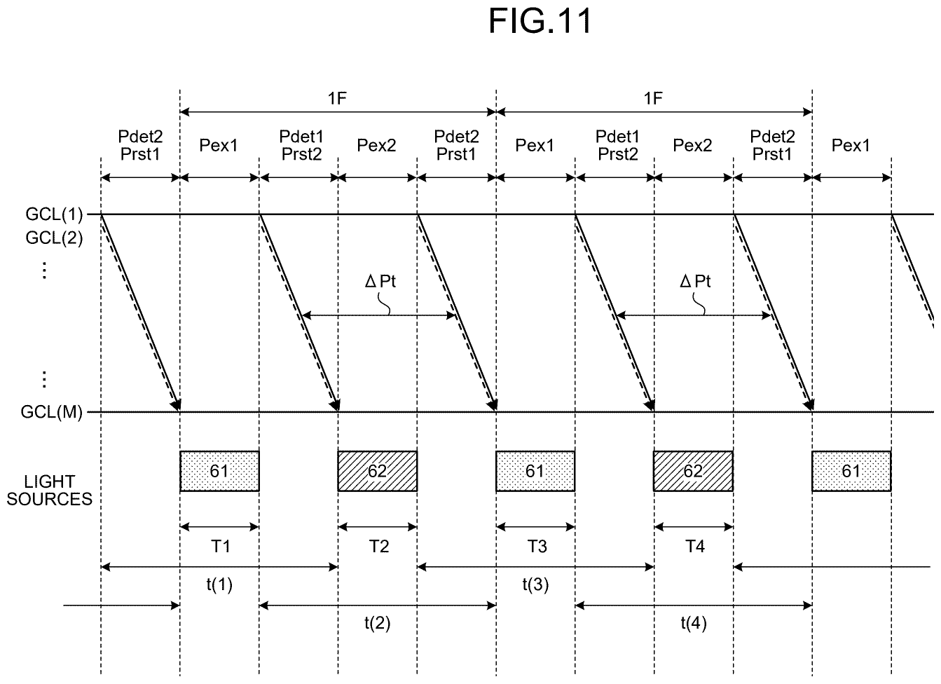

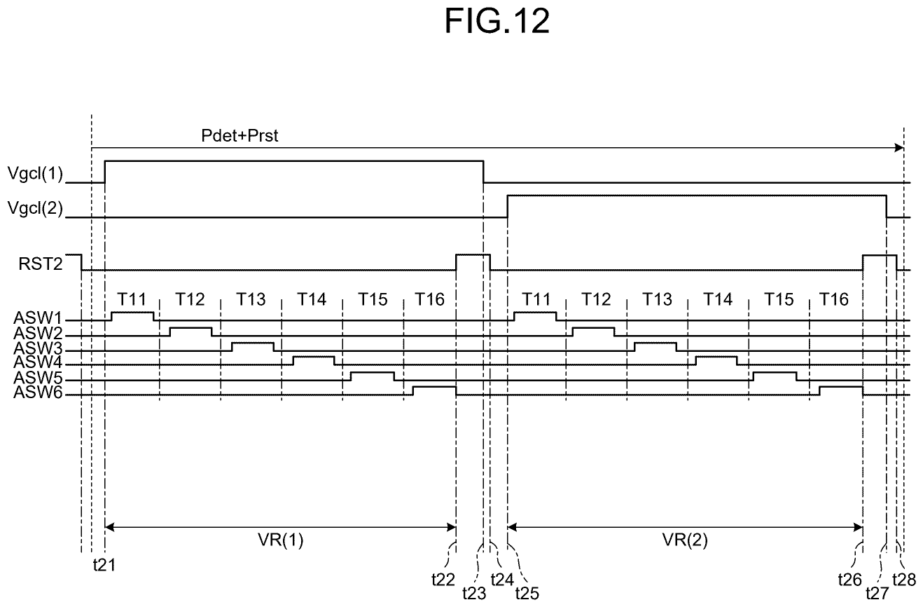

In calculating the blood oxygen saturation level (SpO₂), the pulse wave acquired using the first light and the pulse wave acquired using the second light are used. Therefore, the gap between the detection timing of the first detection value detected based on the first light and the detection timing of the second detection value detected based on the second light is preferably smaller. The following describes an operation example that can reduce the gap between the detection timing of the first detection value detected based on the first light and the detection timing of the second detection value detected based on the second light, with reference to FIGS. 11 and 12.

FIG. 11 is a second explanatory diagram for explaining a second example of the relation between driving of the sensor area and the lighting operations of the light sources in the detection device according to the embodiment. FIG. 12 is a timing waveform diagram illustrating an operation example in the second example illustrated in FIG. 11.

In the second example illustrated in FIG. 11, the first light is the red light, and the second light is the infrared light. In the second example illustrated in FIG. 11, solid line arrows indicate a first reset period Prst1 in the detection operation based on the first light and a second reset period Prst2 in the detection operation based on the second light, and dashed line arrows indicate a first readout period Pdet1 in the detection operation based on the first light and a second readout period Pdet2 in the detection operation based on the second light.

In the second example illustrated in FIG. 11, the detection operation based on the first light is performed in periods t(1), t(3), . . . ; and the detection operation based on the second light is performed in periods t(2), t(4), . . . . Hereinafter, the periods t(1), t(3), . . . during which the detection operation based on the first light is performed are each also referred to as a “first light detection period” and the periods t(2), t(4), . . . during which the detection operation based on the second light is performed are each also referred to as a “second light detection period”. Assuming that the period composed of a first exposure period Pex1 of the first light detection period, the first readout period Pdet1 of the first light detection period, a second exposure period Pex2 of the second light detection period, and the second readout period Pdet2 of the second light detection period corresponds to one frame (1F) unit, the first detection value and the second detection value used to calculate the blood oxygen saturation level (SpO₂) are detected. In the second example illustrated in FIGS. 11 and 12, the light emission period of the first light sources 61 almost coincides with the first exposure period Pex1 in the first light detection period. In the second example illustrated in FIGS. 11 and 12, the light emission period of the second light sources 62 almost coincides with the second exposure period Pex2 in the second light detection period.

In the second example illustrated in FIG. 11, the first reset period Prst1 in the first light detection period and the second readout period Pdet2 in the second light detection period of the previous frame are executed in parallel. In the one frame (1F), the second reset period Prst2 in the second light detection period and the first readout period Pdet1 in the first light detection period are executed in parallel. This way of operation can reduce a gap ΔPt in detection timing between the first and the second detection values used to calculate the blood oxygen saturation level (SpO₂).

In the second example illustrated in FIG. 11, the gate drive signal Vgcl is supplied to the gate line GCL row by row, and the first switching elements Tr belonging to the signal-supplied row are brought into a coupled state. Specifically, as illustrated in FIG. 12, at time t21, the gate line drive circuit 15 supplies the gate drive signal Vgcl(1) at the high-level voltage (power supply voltage VDD) to the gate line GCL(1). The row readout period VR(1) starts at time t21 when the gate drive signal Vgcl(1) becomes the high-level voltage.

Specifically, the control circuit 122 sequentially supplies the selection signals ASW1, . . . , ASW6 to the signal line selection circuit 16 during the period in which the gate drive signal Vgcl(1) is at the high-level voltage (power supply voltage VDD). The third switching elements TrS are sequentially brought into the coupled state in response to the selection signals ASW1, . . . , ASW6. That is, during the period of reading out each row (row readout period VR(1)), when the first switching elements Tr of the signal-supplied row are in the coupled state, the signal line selection circuit 16 couples the signal lines SGL to the AFE circuit 48 column by column in a predetermined order. As a result, the detection signal Vdet of each of the partial detection areas PAA is supplied to the AFE circuit 48.

As illustrated in FIG. 12, in the second example, the selection signals ASW1, . . . , ASW6 are supplied in the order of periods T11, . . . , T16 in a time-division manner. At time t22, the control circuit 122 sets the selection signal ASW6 to the low-level voltage, and the reading of the last column ends. That is, the row readout period VR(1) ends when the selection signal ASW6 has changed to the low-level voltage while gate drive signal Vgcl(1) is at the high-level voltage.

After the completion of the readout period of the given row (row readout period VR(1)) and before the start of the readout period of a row next to the given row (row readout period VR(2)), a reset potential (reference signal COM) is supplied to the optical sensors PD and the signal lines SGL belonging to the given row. Specifically, the control circuit 122 supplies the reset signal RST2 to the reset signal line Lrst at time t22. This operation turns on the fourth switching elements TrR to supply the reference signal COM to the optical sensors PD corresponding to the gate line GCL(1) and the signal lines SGL.

In the second example illustrated in FIG. 12, the time when the reset signal RST2 is set to the high-level voltage coincides with the time when the selection signal ASW6 is set to the low-level voltage, at time t22. However, the timing is not limited thereto. The reset signal RST2 may be set to the high-level voltage after a predetermined period of time has elapsed since the selection signal ASW6 has been set to the low-level voltage.

Then, at time t23, the gate line drive circuit 15 sets the gate drive signal Vgcl(1) to the low-level voltage. This operation brings the first switching elements Tr of the given row into a non-coupled state. At time t24, the control circuit 122 sets the reset signal RST2 to the low-level voltage. This operation ends the readout period Pdet and the reset period Prst of the first row. The capacitance Cb of the AFE circuit 48 is reset by setting the reset switch RSW from the off state through the transition from the on state to the off state between time t22 and t24.

Then, at time t25, the gate line drive circuit 15 supplies the gate drive signal Vgcl(2) at the high-level voltage (power supply voltage VDD) to the gate line GCL(2) of the second row. Subsequently, in the same way as in the first row, the readout period Pdet and the reset period Prst of the second row are executed from time t26 to time t28. The detection for one frame (1F) can be performed by repeating the scanning operation described above to the last row (gate line GCL(256)).

In the second example illustrated in FIGS. 11 and 12, the first reset period Prst1 in the first light detection period (t(1), t(3), . . . ) and the second readout period Pdet2 in the second light detection period of the previous frame are executed in parallel as described above. The second reset period Prst2 in the second light detection period (t(2), t(4), . . . ) and the first readout period Pdet1 in the first light detection period are executed in parallel. Assuming that the period composed of the first exposure period Pex1 of the first light detection period, the first readout period Pdet1 of the first light detection period, the second exposure period Pex2 of the second light detection period, and the second readout period Pdet2 of the second light detection period corresponds to the one frame (1F) unit, the first detection value and the second detection value used to calculate the blood oxygen saturation level (SpO₂) are detected. Thus, in the second example illustrated in FIGS. 11 and 12, the first reset period Prst1 and the second readout period Pdet2 are executed in parallel, and the second reset period Prst2 and the first readout period Pdet1 are executed in parallel. This way of operation can make the gap ΔPt (time difference) between the detection timing of the first detection value and the detection timing of the second detection value smaller than in the first example illustrated in FIG. 10, wherein the first and second detection values are used to calculate the blood oxygen saturation level (SpO₂).

The following describes application examples of the detection device 1 according to the first embodiment.

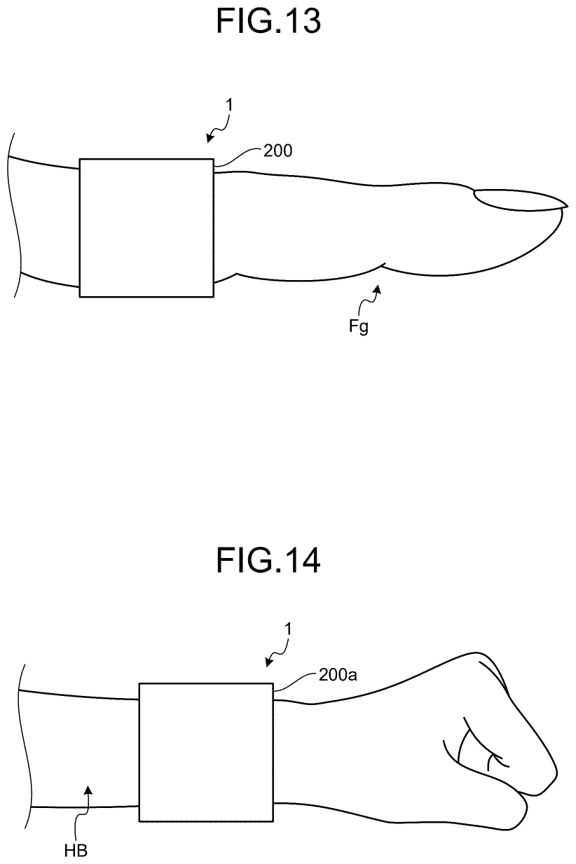

FIG. 13 is a schematic view illustrating a device representing a first application example of the detection device according to the embodiment. A device 200 illustrated in FIG. 13 is a finger-ring-shaped wearable device that can be worn on and removed from a human body and is worn on the finger Fg of the human body. Examples of the finger Fg include a thumb, an index finger, a middle finger, a ring finger, and a little finger. The detection device 1 can detect the biometric information on the living body from the finger Fg wearing the detection device 1.

FIG. 14 is a schematic view illustrating a device representing a second application example of the detection device according to the embodiment. A device 200a illustrated in FIG. 14 serving as the detection device 1 may be, for example, a ring-shaped wearable device such as a smartwatch, a wristwatch, or a wristband. The device 200a is worn on an arm of a human body HB. The human body HB includes wrists, arms, legs, and the like. The detection device 1 can detect the biometric information on the living body from the human body HB wearing the detection device 1.

For the biometric information such as the pulse waves and blood flow, time-domain data in which the detection values acquired by the optical sensor PD are arranged in chronological order needs to be acquired. In the present embodiment, the following describes a specific example of processing that can acquire the chronologically varying biometric information, such as the pulse waves and the blood flow, as the image information in a detection area in each of the devices 200 and 200a to which the detection device 1 having the configuration described above is applied (refer to FIGS. 13 and 14).

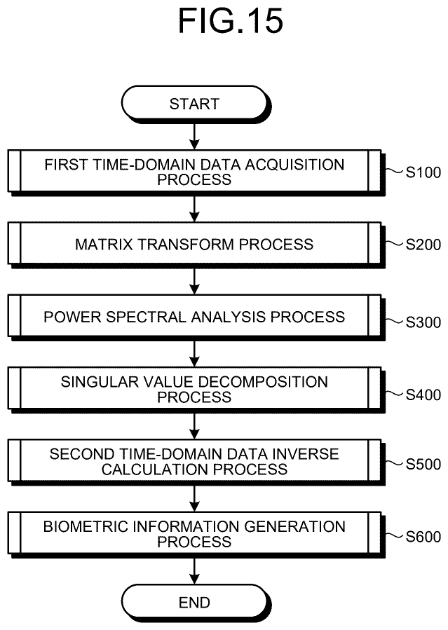

FIG. 15 is a flowchart illustrating an exemplary process in the signal processing circuit of the detection device according to the embodiment.

The signal processing circuit 44 first stores the detection value of each of the optical sensors PD acquired by the AFE circuit 48 during a predetermined period P as first time-domain data in the storage circuit 46 (first time-domain data acquisition process, Step S100).

The predetermined period P for acquiring the detection value of each of the optical sensors PD is set to a length suitable for the pulsation frequency of the biometric information to be detected in the detection device 1. Specifically, when the biometric information to be detected in the detection device 1 is the pulse waves or the blood flow, the predetermined period P for acquiring the detection value of each of the optical sensors PD is, for example, 10 seconds to 20 seconds. When the biometric information to be detected in the detection device 1 is the pulse waves, a pulsation frequency of approximately 1 Hz to 1.5 Hz is exemplified. When the biometric information to be detected in the detection device 1 is the blood flow, a pulsation frequency of approximately 0.05 Hz to 0.15 Hz is exemplified.

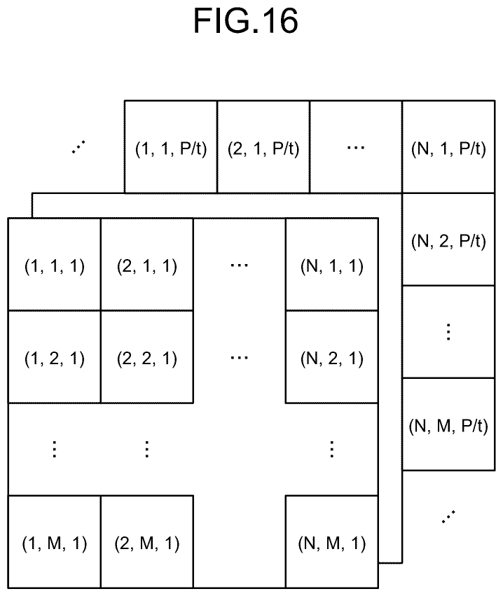

FIG. 16 is an illustrative diagram of the time-domain data acquired in the detection area in the predetermined period. In the example illustrated in FIG. 16, each detection value (n, m, p) corresponding to the optical sensor PD in the nth column of the mth row is obtained at a sampling interval t of the A/D conversion circuit 43 in the predetermined period P (n is a natural number from 1 to N; m is a natural number from 1 to M; and p is a natural number from 1 to P/t).

The signal processing circuit 44 converts the acquired first time-domain data into a time-domain matrix A having elements representing the detection values (n, m, p) arranged in (P/t) columns by (N×M) rows given in Expression (1) below (matrix transform process, Step S200). In the time-domain matrix A, the detection values (n, m, p) are arranged in descending order of time in the row direction, and the detection values (n, m, p) are arranged in the order of spatial arrangement in the column direction. The order of arrangement in the column direction of the detection values (n, m, p) in the time-domain matrix A is not limited to Expression (1) below.

$$A = [ \begin{matrix}( 1 , 1 , 1 ) & … & ( 1 , 1 , P / t ) \\ ( 2 , 1 , 1 ) & … & ( 2 , 1 , P / t ) \\ ⋮ & & ⋮ \\ ( N , 1 , 1 ) & … & ( N , 1 , P / t ) \\ ( 1 , 2 , 1 ) & … & ( 1 , 2 , P / t ) \\ ( 2 , 2 , 1 ) & … & ( 2 , 2 , P / t ) \\ ⋮ & & ⋮ \\ ( N , 2 , 1 ) & … & ( N , 2 , P / t ) \\ ⋮ & & ⋮ \\ ( 1 , M , 1 ) & … & ( 1 , M , P / t ) \\ ( 2 , M , 1 ) & … & ( 2 , M , P / t ) \\ ⋮ & & ⋮ \\ ( N , M , 1 ) & … & ( N , M , P / t )\end{matrix} ] \qquad ( 1 )$$

The signal processing circuit 44 reads the first time-domain data acquired in chronological order for each of the optical sensors PD from the storage circuit 46 and performs a fast Fourier transform (FFT) process on the first time-domain data at each preset unit frequency to calculate a power spectral density (PSD) (power spectral analysis process, Step S300).

Based on the results of the power spectral analysis process, the signal processing circuit 44 performs singular value decomposition (SVD) on the time-domain matrix A given in Expression (1) above, as indicated in Expression (2) below (singular value decomposition process, Step S400).

$$A = {USV}^{T} = ( {u}_{1}^{*} {u}_{2}^{*} … {u}_{K}^{*} ) [ \begin{matrix}{λ}_{1} & … & & 0 \\ ⋮ & {λ}_{2} & & ⋮ \\ & & ⋱ & \\ 0 & … & & {λ}_{K}\end{matrix} ] [ \begin{matrix}{v}_{1}^{*} \\ {v}_{2}^{*} \\ ⋮ \\ {v}_{K}^{*}\end{matrix} ] = {λ}_{1} {u}_{1}^{*} {v}_{1}^{* T} + {λ}_{2} {u}_{2}^{*} {v}_{2}^{* T} + … + {λ}_{K} {u}_{K}^{*} {v}_{K}^{* T} \qquad ( 2 )$$

A matrix S in Expression (2) above represents a K column by K row singular value matrix in which λ₁, λ₂, . . . , AK are arranged as diagonal elements in descending order of the power spectral density obtained by the power spectral analysis process, and the elements other than the diagonal elements are “0”. λₖ is a singular value of the time-domain matrix A that represents the unit frequency in the power spectral analysis process, and a number K of singular values λₖ is a value determined according to the unit frequency in the power spectral analysis process.

U in Expression (2) above indicates a spatial distribution and represents a left singular vector in which elements u′ₖ corresponding to the respective singular values λₖ are arranged in the row direction.

V in Expression (2) above indicates a time distribution and represents a right singular vector in which elements v′ₖ corresponding to the respective singular values λₖ are arranged in the column direction.

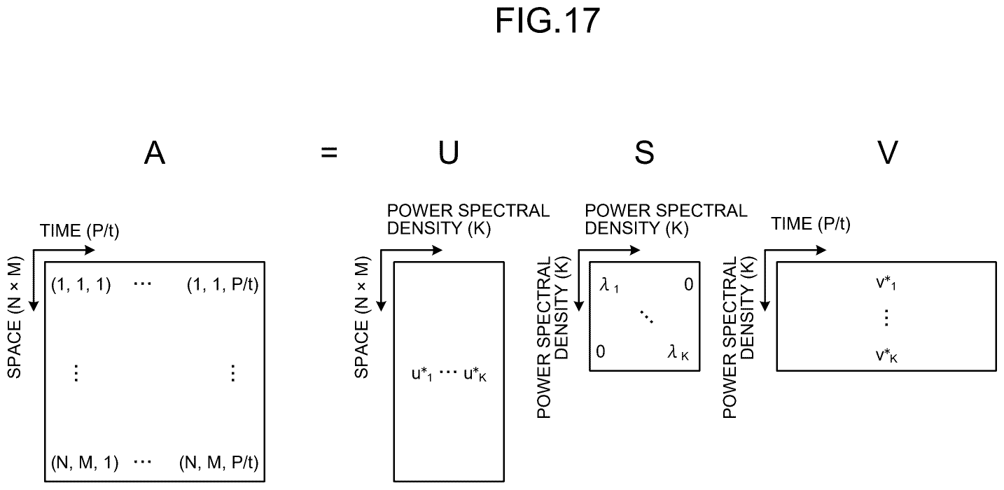

FIG. 17 is a conceptual diagram for explaining an outline of the singular value decomposition process. As illustrated in FIG. 17, the matrix U representing the spatial distribution can be expressed by an orthogonal matrix having K columns and (N×M) rows. A matrix V representing the time distribution can be expressed by an orthogonal matrix having P/t columns and K rows.

In the orthogonal matrix U representing the spatial distribution, the elements u*ₖ corresponding to the respective singular values λₖ are arranged in the row direction in descending order of the power spectral density (u*₁, u*₂, . . . , u*_{K}). In each of the elements u*ₖ of the orthogonal matrix U, spatial components uₖ of fluctuations in components corresponding to the respective singular values λₖ are arranged in the column direction (in space (N×M) direction) in the same order as that in the column direction of the time-domain matrix A.

In the orthogonal matrix V representing the time distribution, the elements v*ₖ corresponding to the respective singular values λₖ are arranged in the column direction in descending order of the power spectral density (v*₁, v*₂, . . . , v*_{K}). In each of the elements v*ₖ of the orthogonal matrix V, time-periodic components vₖ of the fluctuations in the components corresponding to the respective singular values λₖ are arranged in the row direction (time (P/t) direction).

The left-hand side (time-domain matrix A) and the right-hand side (USV^{T}) illustrated in FIG. 17 are interconvertible. In the present disclosure, preset biometric information acquisition conditions are applied to the right-hand side (USV^{T}) after singular value transformation processing.

In the present disclosure, acquisition conditions for acquiring the desired biometric information such as the pulse waves and the blood flow are stored in advance in the storage circuit 46 as the biometric information acquisition conditions. The signal processing circuit 44 reads out the biometric information acquisition conditions stored in the storage circuit 46 and inversely calculates second time-domain data that satisfies the biometric information acquisition conditions using Expressions (1) and (2) given above (second time-domain data inverse calculation process, Step S500).

In Expression (2) given above, the signal processing circuit 44 inversely calculates the second time-domain data based on singular values, which are included in the singular values λ₁, λ₂, . . . , λ_{K} included in the singular value matrix S, that satisfy the biometric information acquisition conditions described above. More specifically, among the singular values λ₁, λ₂, . . . , λ_{K} included in the singular value matrix S, the singular values that do not satisfy the biometric information acquisition conditions are set to “0” while leaving unchanged the singular values that satisfy the above-described biometric information acquisition conditions, and the time-domain matrix A given in Expression (1) above is inversely calculated. The obtained time-domain matrix A is inversely transformed into the second time-domain data in the state illustrated in FIG. 16.

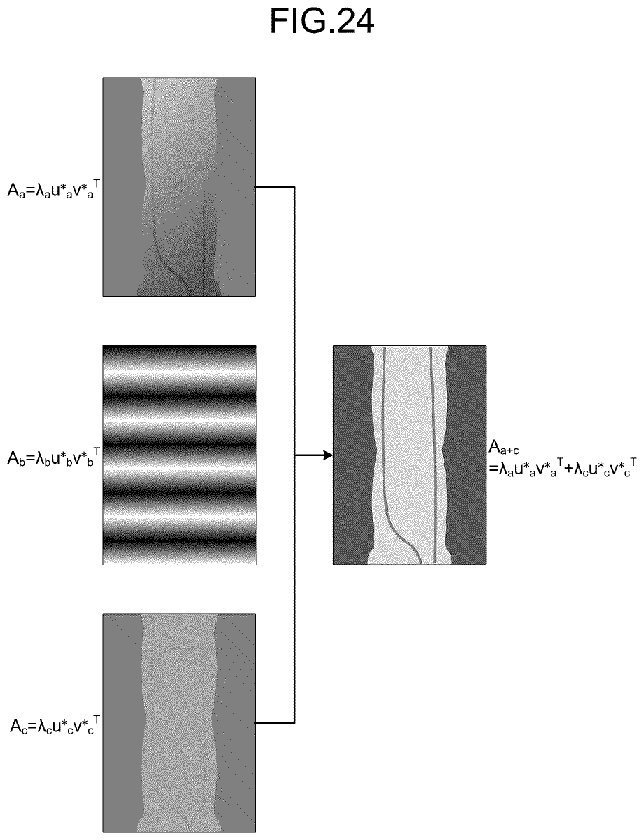

Biometric information acquisition conditions according to characteristics of the human body are set as the biometric information acquisition conditions. For example, as the biometric information acquisition conditions when measuring a blood flow velocity, a frequency range may be set such that the time for phase inversion of outputs at two points determined from the known blood flow velocity for each wearing part falls within a predetermined range, and the singular values corresponding to the other frequencies may be set to “0”. For example, as the biometric information acquisition conditions when measuring the pulse waves, a predetermined frequency range including the frequency of a peak of a waveform may be set, and the singular values corresponding to the other frequencies may be set to “0”. In this case, the predetermined frequency range is set within a range that can be considered as the pulse wave of the human body. Alternatively, for example, a singular value corresponding to a frequency component that can be identified in advance as a noise component may be set to “0”. For example, as the frequency component that can be identified in advance as a noise component, the frequency component of a singular value λ_{b} illustrated in FIG. 24 (to be explained later) can be considered a power supply noise component at a plurality of times the frequency of the one frame period in the detection device 1.

The signal processing circuit 44 uses the second time-domain data of each of the optical sensors PD obtained by the second time-domain data inverse calculation process to generate the biometric information to be detected in the detection device 1 (biometric information generation process, Step S600).

The process described above can remove, for example, noise components other than the biometric information to be detected in the detection device 1, such as the pulse waves and the blood flow (for example, body motion noise caused by human body movement or the like, biosignals not intended to be detected, or noise components at an alternating-current frequency of a commercial power supply (for example, at 50 Hz or 60 Hz)).

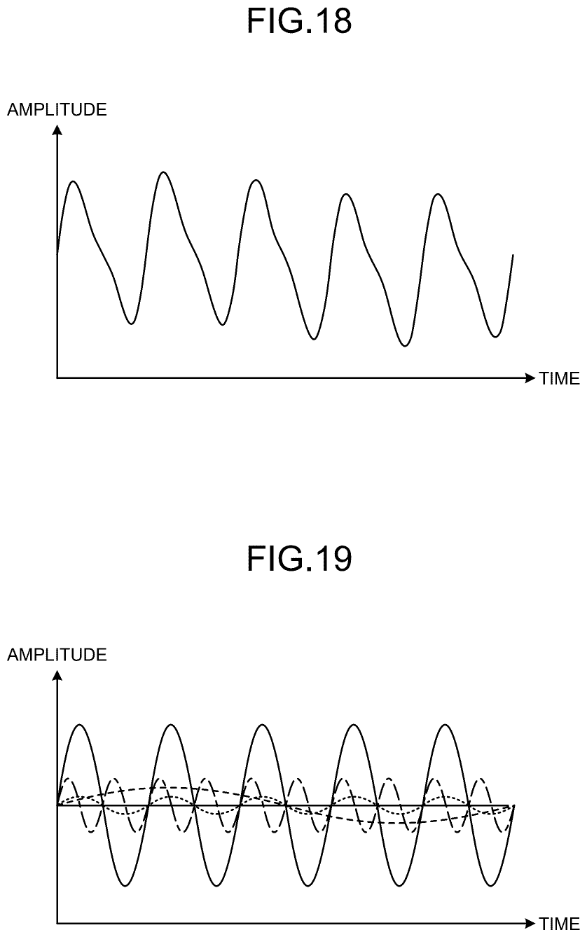

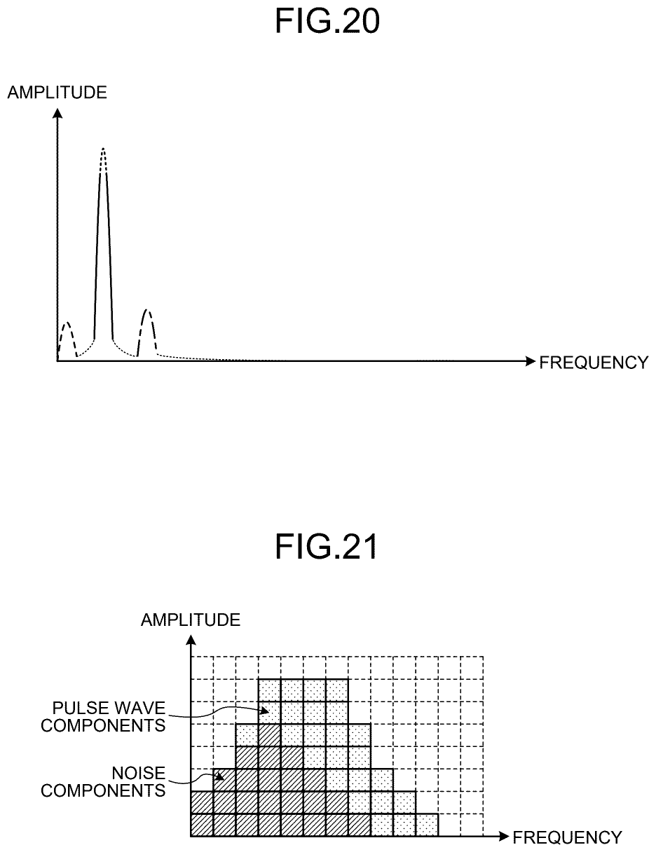

FIG. 18 is a waveform diagram illustrating an exemplary pulse wave. FIG. 19 is an illustrative diagram illustrating exemplary frequency components included in the pulse wave. FIG. 20 is an illustrative diagram illustrating an exemplary frequency distribution obtained by applying the FFT process to the time-domain data constituting the waveform.

As illustrated in FIGS. 18, 19, and 20, the pulse wave includes a plurality of frequency components. When the cycle of the frequency component constituting the pulse wave overlaps the cycle of the noise component other than the biometric information to be detected in the detection device 1, or when the frequency component of the pulse wave is close to the frequency of the noise component, the FFT process may not be able to distinguish the frequency component of the pulse wave from the noise component.

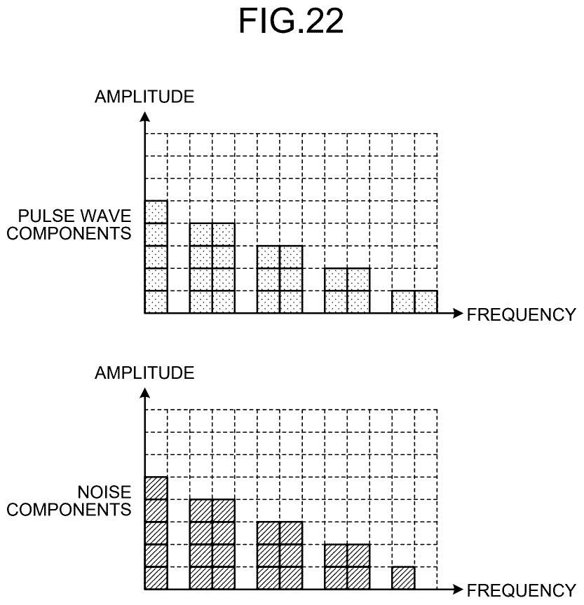

FIG. 21 is an illustrative diagram of the FFT process. FIG. 22 illustrates illustrative diagrams of a process using the singular value decomposition according to the embodiment. FIGS. 21 and 22 illustrate the frequency components of the pulse wave as the biometric information to be detected in the detection device 1.

The FFT process can obtain the magnitude (amplitude) of the frequency components of the pulse wave included in the pulse waveform, but, as illustrated in FIG. 21, may not be able to obtain appropriate amplitude values due to noise components superimposed on the pulse wave components. In addition, phase difference components of the detection values in the detection area, that is, time-series variations of the detection values in the detection area cannot be obtained as the image information.

In contrast, as illustrated in FIG. 22, the above-described process according to the present embodiment can remove the noise components other than the desired frequency components using the singular value decomposition, and can obtain the biometric information to be detected as the image information in the detection area using the time-domain data (second time-domain data) of each of the optical sensors PD after the removal of the noise components. The following describes a concept of obtaining the time-domain data (second time-domain data) of each of the optical sensors PD from which components other than the desired frequency components have been removed, in the above-described process according to the present embodiment.

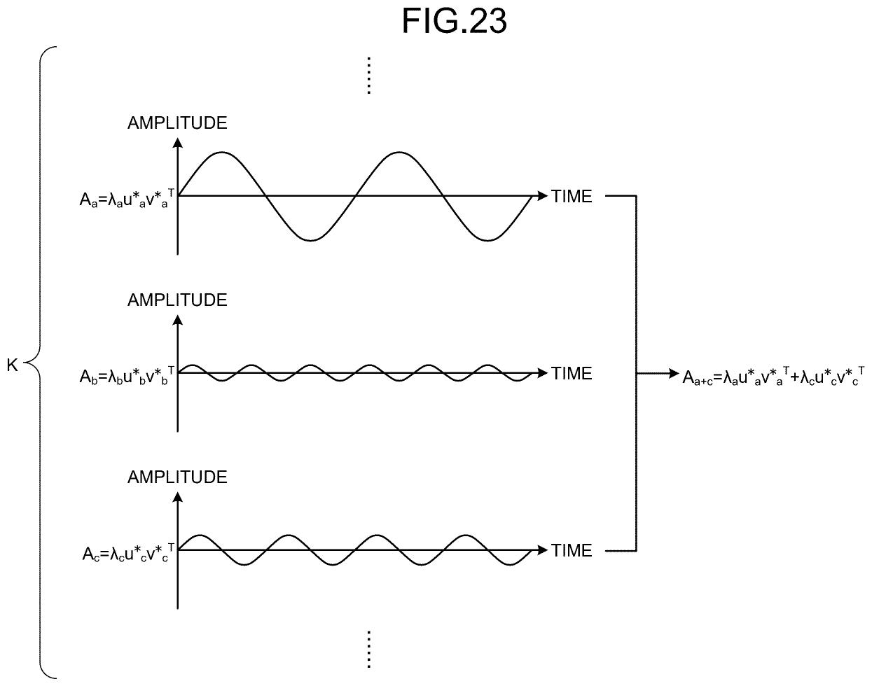

FIG. 23 illustrates illustrative diagrams illustrating exemplary frequency components decomposed by the singular value decomposition process according to the embodiment.

In the above-described process according to the present embodiment, the singular value decomposition process (Step S400 in FIG. 15) is performed so that the first time-domain data (time-domain matrix) is decomposed into K frequency components (where K is the number of the singular values Ak). Then, among the K singular values included in the singular value matrix S, the singular values that do not satisfy the above-described biometric information acquisition conditions are set to “0” while leaving unchanged the singular values that satisfy the biometric information acquisition conditions, and the second time-domain data inverse calculation process (Step S500 in FIG. 15) is performed. Specifically, in the example illustrated in FIG. 23, for example, if the frequency component of a singular value λₐ and the frequency component of a singular value λ_{c} are frequency components that form the biometric information to be detected in the detection device 1, and the frequency components of the other singular values including the singular value λ_{b} are noise components other than the biological information to be detected in the detection device 1, the frequency components of the other singular values including the singular value λ_{b} are set to “0” while leaving unchanged the singular values λₐ and λ_{c}, and the second time-domain data inverse calculation process (Step S500 in FIG. 15) is performed. This process obtains the time-domain data (second time-domain data) from which the frequency components of the other singular values including the singular value λ_{b} that is the noise component have been removed. For example, a time-domain matrix λ_{a+c} obtained by the frequency component of the singular value λₐ and the frequency component of the singular value λ_{c} can be expressed as Expression (3) below.

$${A}_{a + c} = {λ}_{a} {u}_{a}^{⋆} {v}_{a}^{⋆} + {λ}_{c} {u}_{c}^{⋆} {v}_{c}^{⋆} \qquad ( 3 )$$

FIG. 24 illustrates illustrative diagrams illustrating exemplary biometric information acquired as the image information by the detection device according to the embodiment. The illustrative diagrams illustrated in FIG. 24 exemplify the image information obtained by the frequency component of the singular value λₐ and the frequency component of the singular value λ_{c}. The frequency component of the singular value λₐ and the frequency component of the singular value λ_{c} are frequency components from which the frequency component of the singular value λ_{b}, which is the noise component, has been removed and that form the biometric information to be detected in the detection device 1.

The time-domain data (second time-domain data) of each of the optical sensors PD obtained by the second time-domain data inverse calculation process is used to perform the biometric information generation process (Step S600 in FIG. 15). This process can obtain the biometric information from which the frequency component of the singular value λ_{b} that is the noise component has been removed, as the image information in the detection area.

The process described above can obtain the desired biometric information, such as the pulse waves and the blood flow, as the image information in the detection area.

In the embodiment described above, an example of performing the biometric information generation process (Step S600 in FIG. 15) in the signal processing circuit 44 has been described. However, the time-domain data (second time-domain data) of each of the optical sensors PD after the removal of the noise components may be transmitted to the host via the output circuit 126, and the host may generate the biometric information.

While the preferred embodiment has been described above, the present invention is not limited to such an embodiment. The content disclosed in the embodiment is merely an example, and can be variously modified within the scope not departing from the gist of the present invention. Any modifications appropriately made within the scope not departing from the gist of the present invention also naturally belong to the technical scope of the present invention. At least one of various omissions, substitutions, and changes of the components can be made without departing from the gist of the embodiment described above and the modifications thereof.

What is claimed is:

1. A detection device comprising:
a plurality of optical sensors arranged in a detection area;
a light source configured to emit light to the optical sensors;
an analog front-end (AFE) circuit configured to acquire a detection value of each of the optical sensors; and
a signal processing circuit configured to acquire predefined biometric information based on first time-domain data obtained by acquiring the detection values in chronological order, wherein
the signal processing circuit is configured to:
convert the first time-domain data into a time-domain matrix and perform singular value decomposition on the time-domain matrix, and inversely calculate second time-domain data based on a predetermined singular value among a plurality of singular values obtained as a result of the singular value decomposition; and
acquire the biometric information that changes in chronological order as image information using the second time-domain data.

2. The detection device according to claim 1, wherein
the optical sensors are organic photodiodes and each comprise:
an active layer;
an upper electrode provided with an upper buffer layer interposed between the active layer and the upper electrode; and
a lower electrode provided with a lower buffer layer interposed between the active layer and the lower electrode.

3. The detection device according to claim 1, wherein
the signal processing circuit is configured to:
perform a power spectral analysis process on the first time-domain data; and
generate a singular value matrix in which the singular values are arranged as diagonal elements in descending order of power spectral density obtained by the power spectral analysis process.

4. The detection device according to claim 3, wherein the signal processing circuit is configured to generate, in the singular value decomposition, a left singular vector indicating a spatial distribution in which elements corresponding to the respective singular values are arranged in a row direction and a right singular vector indicating a time distribution in which the elements corresponding to the respective singular values are arranged in a column direction.

5. The detection device according to claim 4, wherein when A denotes the time-domain matrix; S denotes the singular value matrix; U denotes the left singular vector; V denotes the right singular vector; λₖ denotes a singular value that is each element of the singular value matrix S (k is a natural number from 1 to K); u*ₖ denotes each element of the left singular vector U; and v*ₖ denotes each element of the right singular vector V, the singular value decomposition is expressed using Expression (1) below.
$$A = {USV}^{T} = ( {u}_{1}^{*} {u}_{2}^{*} … {u}_{K}^{*} ) [ \begin{matrix}{λ}_{1} & … & & 0 \\ ⋮ & {λ}_{2} & & ⋮ \\ & & ⋱ & \\ 0 & … & & {λ}_{K}\end{matrix} ] [ \begin{matrix}{v}_{1}^{*} \\ {v}_{2}^{*} \\ ⋮ \\ {v}_{K}^{*}\end{matrix} ] = {λ}_{1} {u}_{1}^{*} {v}_{1}^{* T} + {λ}_{2} {u}_{2}^{*} {v}_{2}^{* T} + … + {λ}_{K} {u}_{K}^{*} {v}_{K}^{* T} \qquad ( 1 )$$

6. The detection device according to claim 5, wherein the signal processing circuit is configured to
set, to zero, singular values that do not satisfy preset biometric information acquisition conditions among K singular values included in the singular value matrix, and
inversely calculate the second time-domain data.

7. The detection device according to claim 1, wherein the light source comprises at least a first light source configured to emit first light to the optical sensors.

8. The detection device according to claim 7, wherein the first light is red light or infrared light.

9. The detection device according to claim 7, wherein the first light is blue light or green light.

10. The detection device according to claim 1, wherein
the light source comprises:
a first light source configured to emit first light to the optical sensors; and
a second light source configured to emit second light to the optical sensors.

11. The detection device according to claim 10, wherein
the first light is red light, and
the second light is infrared light.

12. A wearable device comprising the detection device as claimed in claim 1, the wearable device having a ring shape wearable on a human body.

13. The wearable device according to claim 12, configured to be worn on a finger or a thumb of the human body.

14. The wearable device according to claim 12, configured to be worn on a wrist or an arm of the human body.

15. The wearable device according to claim 12, configured to be worn on a leg of the human body.
